(19) United States
(12) Patent Application Publication
(10) Pub. No.: US 2010/0209926 A1
(43) Pub. Date: Aug. 19, 2010

(54) METHODS FOR DETECTING NEUTRALIZING ANTIBODIES FOR BONE MORPHOGENETIC PROTEINS

(75) Inventors: Hicham Alaoui, Hopkinton, MA (US); Karen Lavery, Franklin, MA (US)

Correspondence Address: ROPES & GRAY LLP, PATENT DOCKETING 39/361, 1211 AVENUE OF THE AMERICAS, NEW YORK, NY, 10036-8704, US

(73) Assignee: Stryker Corporation, Kalamazoo, MI (US)

(21) Appl. No.: 12/679,965

(22) PCT Filed: Sep. 26, 2008
(86) PCT No.: PCT/US08/11154

§ 371 Date: Apr. 6, 2010

Related U.S. Application Data
(60) Provisional application No. 60/995,833, filed on Sep. 28, 2007.

(51) Int. Cl. C12Q 1/68

(52) U.S. Cl. 435/6

(57) ABSTRACT

The present invention relates to methods of detecting neutralizing antibodies for bone morphogenetic proteins (BMP). More particularly, it relates to a highly specific, robust, rapid and accurate cell-based assay for detecting the presence of anti-BMP neutralizing antibodies.

### FIELD OF THE INVENTION

The present invention relates to methods of detecting neutralizing antibodies for bone morphogenetic proteins (BMP). More particularly, it relates to a highly specific, robust, rapid and accurate cell-based assay for detecting the presence of anti-BMP neutralizing antibodies.

### BACKGROUND OF THE INVENTION

Osteogenic and chondrogenic proteins are able to induce the proliferation and differentiation of progenitor cells into functional bone, cartilage, tendon, and/or ligamentous tissue. These proteins, referred to herein as “osteogenic proteins,” “morphogenic proteins,” “morphogenetic proteins” or “morphogens,” include members of the bone morphogenetic protein (“BMP”) family identified by their ability to induce endochondral bone morphogenesis. The osteogenic proteins generally are classified in the art as a subgroup of the TGF-β superfamily of growth factors. Hogan, Genes & Development 10:1580-1594 (1996). Osteogenic proteins include the mammalian osteogenic protein-1 (OP-1, also known as BMP-7) and its Drosophila homolog 60A, osteogenic protein-2 (OP-2, also known as BMP-8), osteogenic protein-3 (OP-3), BMP-2 (also known as BMP-2A or CBMP-2A) and its Drosophila homolog DPP, BMP-3, BMP-4 (also known as BMP-2B or CBMP-2B), BMP-5, BMP-6 and its murine homolog Vgr-1, BMP-9, BMP-10, BMP-11, BMP-12, GDF-3 (also known as Vgr2), GDF-8, GDF-9, GDF-10, GDF-11, GDF-12, BMP-13, BMP-14, BMP-15, GDF-5 (also known as CDMP-1 or MP52), GDF-6 (also known as CDMP-2), GDF-7 (also known as CDMP-3), the Xenopus homolog Vgl and NODAL, UNIVIN, SCREW, ADMP, and NEURAL.

Osteogenic proteins typically include secretory peptides sharing common structural features. Processed from a precursor “pro-form,” the mature form of an osteogenic protein is a disulfide-bonded homo- or hetero-dimer, with each subunit having a carboxyl terminal active domain. This domain has approximately 97-106 amino acid residues and contains a conserved pattern of cysteine residues. See, e.g., Massague, Annu. Rev. Cell Biol. 6:597 (1990); Sampath et al., J. Biol. Chem. 265:13198 (1990).

Osteogenic proteins can regulate numerous biological processes, including the induction of new bone and cartilage formation in both developing and mature skeletal systems. For example, osteogenic proteins can stimulate the proliferation and differentiation of progenitor cells when administered to a mammal. As a result, they can induce bone formation, including endochondral bone formation, under conditions where true replacement bone would not otherwise occur. Osteogenic proteins can also induce formation of new bone in large segmental bone defects, spinal fusions, and fractures.

Detection of particular antibodies is a very common form of medical diagnostics. For example, in biochemical assays for disease diagnosis, a titer of antibodies directed against any particular antigen can be estimated from the blood. In clinical immunology, the levels of different classes of immunoglobulins are sometimes useful to characterize the antibody profile of patients in determining the cause of diseases.

Detection of antibodies, such as neutralizing antibodies, can also be used to monitor the development of potential immunogenicity in patients treated with a therapeutic agent. For example, neutralizing antibodies in patients treated with OP-1 (BMP-7) for revision posterolateral spine fusions and treatment of long bone non unions are currently detected using several immunodiagnostic methods based on detection of complex antigen-antibody, including, for example, enzyme-linked immunosorbent assay (ELISA), receptor binding assay, radio-immunoprecipitation, biosensor-based assay, immunofluorescence, Western blot, immunodiffusion, and immunoelectrophoresis. Neutralizing antibodies can also be detected using various cell-based systems. In these cell-based assays, neutralizing antibodies inhibit the ability of the therapeutic agent to modulate a biological process in the target cell. These assays may involve, for example, the activation of a reporter gene, such as luciferase. Alternatively, neutralizing antibodies can be detected using cell-based systems involving a biological functional readout, such as alkaline phosphatase activity. However, these current detection methods suffer from a number of drawbacks including the level of sensitivity, the level of specificity as well as the lengthy duration of the assays.

Therefore, there remains a need for an improved method for detecting the presence of anti-BMP neutralizing antibodies which overcome the problems associated with the currently available methods.

### SUMMARY OF THE INVENTION

The present invention solves the above problem by providing a highly specific, robust, rapid and accurate assay for detecting the presence of anti-BMP neutralizing antibodies. Accordingly, in some embodiments, the invention provides a method for the detection of neutralizing antibodies to a bone morphogenetic protein (BMP) in a sample comprising the steps of: (a) contacting said sample with a BMP; (b) incubating said sample from step (a) with a BMP-responsive cell comprising at least one endogenous gene, the expression of which is capable of being modulated by exposure to said BMP; (c) isolating mRNA from said cell; (d) preparing cDNA corresponding to said mRNA by reverse transcription; (e) amplifying said gene from said cDNA of step (d) by quantitative real-time polymerase chain reaction (QPCR); (f) determining the amount of said gene amplified in step (e) in said cells; (g) determining the amount of said gene amplified according to step (e) in control cells contacted with BMP alone; and (h) detecting the presence of neutralizing antibodies in said sample if the amount determined in step (f) is less than or greater than the amount determined in step (g). In some embodiments, the amount determined in step (f) is greater than the amount determined in step (g). In other embodiments, the amount determined in step (f) is less than the amount determined in step (g).

In some embodiments, the invention provides a method for the detection of neutralizing antibodies to a bone morphogenetic protein (BMP) in a sample further comprising the steps of: (i) amplifying a housekeeping gene from said cDNA of step (d) by quantitative real-time polymerase chain reaction (QPCR); (j) determining the amount of said housekeeping gene amplified in step (i) in said cells; and (k) normalizing the amount of said gene determined in step (f) with the amount of said housekeeping gene determined in step (j).

In some embodiments, the bone morphogenetic protein includes, but is not limited to, OP-1 (BMP-7), OP-2, OP-3, BMP-2, BMP-3, BMP-4, BMP-5, BMP-6, BMP-8, BMP-9, BMP-10, BMP-11, BMP-12, BMP-13, BMP-15, BMP-16, BMP-17, BMP-18, DPP, Vg1, Vgr, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, NODAL, UNIVIN, SCREW, ADMP, NEURAL, and fragments thereof. In other embodiments, the bone morphogenetic protein is OP-1 (BMP-7).

In some embodiments, the sample is serum. In other embodiments, the sample is human serum.

In some embodiments, the BMP-responsive cell includes, but is not limited to, A549, SAOS-2, ROS and CSC12. In other embodiments, the BMP-responsive cell is a A549 cell. In some embodiments, the BMP-responsive cell includes, but is not limited to, primary stromal cells derived from either bone marrow, fat or muscle.

In some embodiments, the endogenous gene, the expression of which is capable of being modulated by exposure to a BMP includes, but is not limited to, a gene selected from a gene set forth in Table 1 (see infra).

In some embodiments, the endogenous gene, the expression of which is capable of being modulated by exposure to a BMP is an early gene. In some embodiments, an early gene is a gene whose expression is modulated within a minute up to 48 hours following exposure to a BMP. In some embodiments, the early gene includes, but is not limited to, Id-1, Id-2, Id-3, Id-4, Msx2, Dlx2, Dlx3, Dlx5, Noggin, Smad6, Smad7 and Runx2. In some embodiments, the early gene is Id-1.

In some embodiments, the endogenous gene, the expression of which is capable of being modulated by exposure to a BMP is a late gene. In some embodiments, a late gene is a gene whose expression is modulated more than 48 hours after exposure to a BMP. In some embodiments, the late genes includes but is not limited to HEY1, DIO2, ADAMTS9, HAS3, FGFR3, MFI2, CHI3L1, NOG, BAMBI, GREM1, GREM2 and SOST.

In some embodiments, the expression of the gene is up-regulated by exposure to a BMP. In other embodiments, the expression of the gene is down-regulated by exposure to a BMP.

In some embodiments, the sample is contacted with a BMP in step (a) for at least 30 minutes.

In some embodiments, the sample from step (b) is incubated with a BMP-responsive cell for about 3 hours.

In some embodiments, the antibody is specific for OP-1 (BMP-7).

In some embodiments, the sample is subjected to at least one prescreening assay prior to performing steps (a)-(h) of the method. In some embodiments, the prescreening assay is any assay that is capable of detecting neutralizing antibodies to a bone morphogenetic protein (BMP). In some embodiments, the prescreening assay is selected from the group consisting of immunoassay and cell-based assay.

In some embodiments, the immunoassay is an enzyme-linked-immunosorbent assay (ELISA).

In some embodiments, the cell-based assay involves activation of a reporter gene. In some embodiments, the reporter gene is a luciferase gene. In some embodiments, the luciferase gene is linked to a promoter of a gene that is capable of being modulated by exposure to a BMP. In some embodiments, the gene is an early or late gene. In some embodiments, the gene includes, but is not limited to, Id-1, Id-2, Id-3, Id-4, Msx2, Dlx2, Dlx3, Dlx5, Noggin, Smad6, Smad7, Runx2, fibromodulin, Hey 1 and SFRP-2.

In some embodiments, the cell-based assay involves monitoring the activity of alkaline phosphatase. In some embodiments, the alkaline phosphatase activity is monitored in rat osteosarcoma (ROS) cells.

### BRIEF DESCRIPTION OF THE DRAWINGS

FIG. 1 shows that the effective concentration of BMP-7 at which greater assay sensitivity was achieved is 15 ng/mL. Two different concentrations (15 ng/mL and 25 ng/mL) of BMP-7 (each done in triplicate) were incubated with increasing concentrations of an anti-BMP-7 monoclonal antibody (12G3). The relative quantity (RQ) values for the Id-1 gene were determined and BMP-7 at a concentration of 15 ng/mL was determined to be the effective concentration at which greater assay sensitivity was achieved.

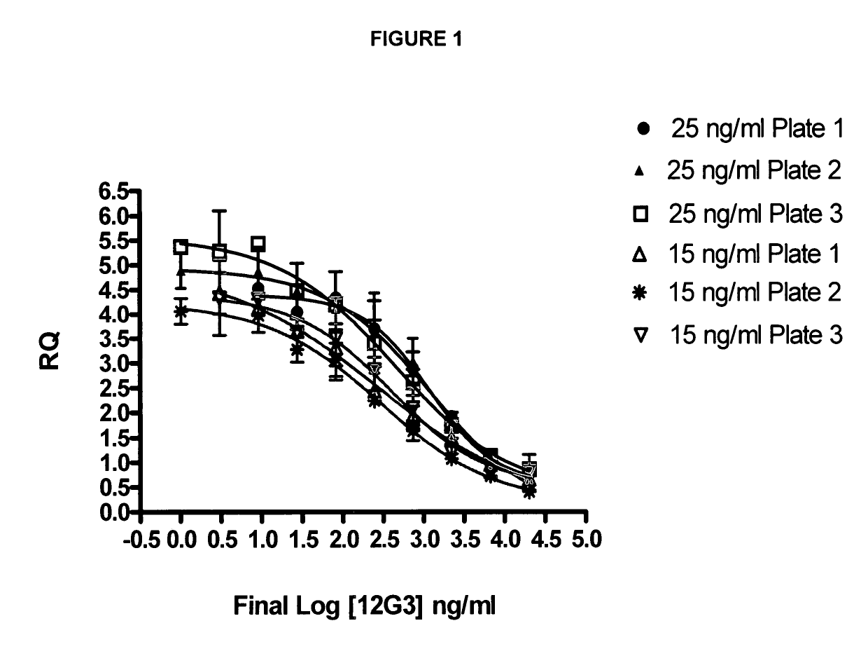

FIG. 2 shows the minimum required dilution (MRD) of serum samples to be 1:40. Normal human serum (NHS) samples were diluted to 1:10, 1:20, 1:40 and 1:80 and the relative quantity (RQ) values for the Id-1 gene were determined. The MRD is the minimum dilution at which the serum samples display background RQ value similar to control samples containing no serum (No NHS).

FIG. 3 shows the assay optimization of the time period of incubation and concentration of FBS. Cells were incubated for either 19 hours or 3 hours with increasing concentrations of BMP-7 and the effect on the ratio of target/housekeeping (Id-1/GAPDH) gene expression was determined. The 3 hour time period of incubation was chosen as the optimized time period based on the response curve.

FIG. 4 shows that OP-1 (BMP-7) does not affect the housekeeping GAPDH expression ratio. The GAPDH expression ratio (unspiked/spiked with OP-1) of eighty (80) different samples were studied and shown to be relatively uniform.

FIG. 5 shows the fold change in mRNA expression of FGFR3, DIO2, HEY1, ADAMTS9, HAS3, and MFI2 following treatment with 40 ng/ml (BMP-7 40) and 400 ng/ml (BMP-7 400) of BMP-7 over a 7 day period. ODM refers to mRNA from control cells (i.e., no BMP treatment).

FIG. 6 shows the fold change in mRNA expression of Noggin, BAMBI, GREM1, GREM2 and SOST following treatment with 40 ng/ml (BMP-7 40) and 400 ng/ml (BMP-7 400) of BMP-7 over a 7 day period. ODM refers to mRNA from control cells (i.e., no BMP treatment).

FIG. 7 shows the fold change in mRNA expression of CHI3L1 following treatment with 40 ng/ml (BMP-7 40) and 400 ng/ml (BMP-7 400) of BMP-7 over a 7 day period. ODM refers to mRNA from control cells (no BMP treatment).

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

In order to further define the invention, the following terms and definitions are provided herein.

The terms “bone morphogenetic protein (BMP),” “bone morphogenic protein (BMP),” “morphogenic protein” or “morphogenetic protein” are used interchangeably herein and refer to a protein belonging to the BMP family of the TGF-β superfamily of proteins (BMP family) based on DNA and amino acid sequence homology. A protein belongs to the BMP family according to this invention when it has at least 50% amino acid sequence identity with at least one known BMP family member within the conserved C-terminal cysteine-rich domain, which characterizes the BMP protein family. Preferably, the protein has at least 70% amino acid sequence identity with at least one known BMP family member within the conserved C-terminal cysteine rich domain. Members of the BMP family may have less than 50% DNA or amino acid sequence identity overall. BMPs may be capable of inducing progenitor cells to proliferate and/or to initiate differentiation pathways that lead to cartilage, bone, tendon, ligament, kidney, liver, muscle, neural or other types of tissue formation depending on local environmental cues, and thus BMPs may behave differently in different surroundings. For example, a BMP may induce bone tissue at one treatment site and neural tissue at a different treatment site.

Bone morphogenetic proteins useful in the practice of the invention include active forms of OP-1, OP-2, OP-3, BMP-2, BMP-3, BMP-4, BMP-5, BMP-6, BMP-8, BMP-9, BMP-10, BMP-11, BMP-12, BMP-13, BMP-15, BMP-16, BMP-17, BMP-18, DPP, Vg1, Vgr-1, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, UNIVIN, NODAL, SCREW, ADMP or NEURAL, and amino acid sequence variants thereof.

The term “amino acid sequence homology” is understood to include both amino acid sequence identity and similarity. Homologous sequences share identical and/or similar amino acid residues, where similar residues are conservative substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence. Certain particularly preferred morphogenic polypeptides share at least 60%, and preferably 70% amino acid sequence identity with the C-terminal 102-106 amino acids, defining the conserved seven-cysteine domain of human OP-1 and related proteins.

Amino acid sequence homology can be determined by methods well known in the art. For instance, to determine the percent homology of a candidate amino acid sequence to the sequence of the seven-cysteine domain, the two sequences are first aligned. The alignment can be made with, e.g., the dynamic programming algorithm described in Needleman et al., J. Mol. Biol., 48, pp. 443 (1970), and the Align Program, a commercial software package produced by DNAstar, Inc. The teachings by both sources are incorporated by reference herein. An initial alignment can be refined by comparison to a multi-sequence alignment of a family of related proteins. Once the alignment is made and refined, a percent homology score is calculated. The aligned amino acid residues of the two sequences are compared sequentially for their similarity to each other. Similarity factors include similar size, shape and electrical charge. One particularly preferred method of determining amino acid similarities is the PAM250 matrix described in Dayhoff et al., Atlas of Protein Sequence and Structure, 5, pp. 345-352 (1978 & Supp.), which is incorporated herein by reference. A similarity score is first calculated as the sum of the aligned pair wise amino acid similarity scores. Insertions and deletions are ignored for the purposes of percent homology and identity. Accordingly, gap penalties are not used in this calculation. The raw score is then normalized by dividing it by the geometric mean of the scores of the candidate sequence and the seven-cysteine domain. The geometric mean is the square root of the product of these scores. The normalized raw score is the percent homology.

The term “conservative substitutions” refers to residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., supra. Examples of conservative substitutions are substitutions within the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. The term “conservative variant” or “conservative variation” also includes the use of a substituting amino acid residue in place of an amino acid residue in a given parent amino acid sequence, where antibodies specific for the parent sequence are also specific for, i.e., “cross-react” or “immuno-react” with, the resulting substituted polypeptide sequence.

The term “osteogenic protein (OP)” refers to a morphogenic protein that is capable of inducing a progenitor cell to form cartilage and/or bone. The bone may be intramembraneous bone or endochondral bone. Most osteogenic proteins are members of the BMP protein family and are thus also BMPs. As described elsewhere herein, the class of proteins is typified by human osteogenic protein (hOP-1). Osteogenic proteins suitable for use with the present invention can be identified by means of routine experimentation using the art-recognized bioassay described by Reddi and Sampath (Sampath et al., Proc. Natl. Acad. Sci., 84, pp. 7109-13, incorporated herein by reference).

The terms “morphogenic activity,” “morphogenetic activity,” “inducing activity” and “tissue inductive activity” alternatively refer to the ability of a bone morphogenetic protein to stimulate a target cell to undergo one or more cell divisions (proliferation) that may optionally lead to cell differentiation. Such target cells are referred to generically herein as progenitor cells. Cell proliferation is typically characterized by changes in cell cycle regulation and may be detected by a number of means which include measuring DNA synthetic or cellular growth rates. Early stages of cell differentiation are typically characterized by changes in gene expression patterns relative to those of the progenitor cell, which may be indicative of a commitment towards a particular cell fate or cell type. Later stages of cell differentiation may be characterized by changes in gene expression patterns, cell physiology and morphology. Any reproducible change in gene expression, cell physiology or morphology may be used to assess the initiation and extent of cell differentiation induced by a BMP.

The term “antibody” refers to a full antibody, e.g., an antibody comprising two heavy chains and two light chains, or to an antigen-binding fragment of a full antibody, and encompasses any polypeptide comprising an antigen-binding site regardless of the source, species of origin, method of production, and characteristics. As a non-limiting example, the term “antibody” includes human, orangutan, mouse, rat, goat, sheep, and chicken antibodies. The term includes, but is not limited to, polyclonal, monoclonal, mono-specific, poly-specific, non-specific, humanized, single-chain, chimeric, synthetic, recombinant, hybrid, mutated, and CDR-grafted antibodies. The term “antibody” also includes, but is not limited to, antibody fragments produced by digestion with various proteases, those produced by chemical cleavage and/or chemical dissociation, and those produced recombinantly. Among these fragments are Fab, Fab′, F(ab′)₂, Fv, scFv, Fd, dAb, and other antibody fragments that retain the antigen-binding function. The antibody or fragment thereof may be any of the known antibody isotypes and their conformations, for example, IgA, IgG, IgD, IgE, IgM monomers, IgA dimers, IgA trimers, or IgM pentamers.

The term “neutralizing antibody” refers to any antibody or fragment thereof capable of binding to and interfering with at least one biological activity of the molecule for which the antibody is specific. The neutralizing antibody may inhibit (i.e., eliminate or reduce) one or more activities of the molecule for which the antibody is specific without inhibiting other activities of the molecule.

The term “modulate” or “modulating” refers to the ability to up-regulate or down-regulate at least one activity of another compound or molecule, such as, for example, the level of gene transcription or the level of gene expression.

The term “BMP-responsive cell” refers to any cell that expresses BMP receptors and allows the transduction of BMP-induced protein signaling, resulting in the modulation of gene expression regulatory elements in response to the interaction between BMP and the cell receptors.

The term “subject” refers to an animal. In some embodiments, the animal is a mammal, including but not limited to a human, bovine and rodent. In other embodiments, the mammal is a human.

### Bone Morphogenetic Protein Family

The BMP family, named for its representative bone morphogenetic/osteogenic protein family members, belongs to the TGF-β protein superfamily. Of the reported “BMPs” (BMP-1 to BMP-18), isolated primarily based on sequence homology, all but BMP-1 remain classified as members of the BMP family of morphogenetic proteins (Ozkaynak et al., EMBO J., 9, pp. 2085-93 (1990)).

The BMP family includes other structurally-related members which are morphogenetic proteins, including the drosophila decapentaplegic gene complex (DPP) products, the Vg1 product of Xenopus laevis and its murine homolog, Vgr-1 (see, e.g., Massagué, Annu. Rev. Cell Biol., 6, pp. 597-641 (1990), incorporated herein by reference).

The C-terminal domains of BMP-3, BMP-5, BMP-6, and OP-1 (BMP-7) are about 60% identical to that of BMP-2, and the C-terminal domains of BMP-6 and OP-1 are 87% identical. BMP-6 is likely the human homolog of the murine Vgr-1 (Lyons et al., Proc. Natl. Acad. Sci. U.S.A., 86, pp. 4554-59 (1989)); the two proteins are 92% identical overall at the amino acid sequence level (U.S. Pat. No. 5,459,047, incorporated herein by reference). BMP-6 is 58% identical to the Xenopus Vg-1 product.

The naturally occurring bone morphogenetic proteins share substantial amino acid sequence homology in their C-terminal regions (domains). Typically, the naturally occurring osteogenic proteins are translated as a precursor, having an N-terminal signal peptide sequence typically less than about 30 residues, followed by a “pro” domain that is cleaved to yield the mature C-terminal domain of approximately 97-106 amino acids. The signal peptide is cleaved rapidly upon translation, at a cleavage site that can be predicted in a given sequence using the method of Von Heijne Nucleic Acids Research, 14, pp. 4683-4691 (1986). The pro domain typically is about three times larger than the fully processed mature C-terminal domain.

Another characteristic of the BMP protein family members is their apparent ability to dimerize. Several bone-derived osteogenic proteins (OPs) and BMPs are found as homo- and heterodimers in their active forms. The ability of OPs and BMPs to form heterodimers may confer additional or altered morphogenetic inductive capabilities on bone morphogenetic proteins. Heterodimers may exhibit qualitatively or quantitatively different binding affinities than homodimers for OP and BMP receptor molecules. Altered binding affinities may in turn lead to differential activation of receptors that mediate different signaling pathways, which may ultimately lead to different biological activities or outcomes. Altered binding affinities could also be manifested in a tissue or cell type-specific manner, thereby inducing only particular progenitor cell types to undergo proliferation and/or differentiation.

In preferred embodiments, the pair of osteogenic or morphogenic polypeptides have amino acid sequences each comprising a sequence that shares a defined relationship with an amino acid sequence of a reference bone morphogenetic protein. Herein, preferred osteogenic or morphogenic polypeptides share a defined relationship with a sequence present in active human OP-1, SEQ ID NO: 1. However, any one or more of the naturally occurring or biosynthetic sequences disclosed herein similarly could be used as a reference sequence. Preferred osteogenic or morphogenic polypeptides share a defined relationship with at least the C-terminal six cysteine domain of human OP-1, residues 335-431 of SEQ ID NO: 1. Preferably, osteogenic or morphogenic polypeptides share a defined relationship with at least the C-terminal seven cysteine domain of human OP-1, residues 330-431 of SEQ ID NO: 1. That is, preferred polypeptides in a dimeric protein with bone morphogenetic activity each comprise a sequence that corresponds to a reference sequence or is functionally equivalent thereto.

Functionally equivalent sequences include functionally equivalent arrangements of cysteine residues disposed within the reference sequence, including amino acid insertions or deletions which alter the linear arrangement of these cysteines, but do not materially impair their relationship in the folded structure of the dimeric bone morphogenetic protein, including their ability to form such intra- or inter-chain disulfide bonds as may be necessary for morphogenic activity. Functionally equivalent sequences further include those wherein one or more amino acid residues differs from the corresponding residue of a reference sequence, e.g., the C-terminal seven cysteine domain (also referred to herein as the conserved seven cysteine skeleton) of human OP-1, provided that this difference does not affect its biological activity. Accordingly, conservative substitutions of corresponding amino acids in the reference sequence are preferred. Amino acid residues that are conservative substitutions for corresponding residues in a reference sequence are those that are physically or functionally similar to the corresponding reference residues, e.g., that have similar size, shape, electric charge, chemical properties including the ability to form covalent or hydrogen bonds, or the like. Particularly preferred conservative substitutions are those fulfilling the criteria defined for an accepted point mutation in Dayhoff et al., supra, the teachings of which are incorporated by reference herein.

The osteogenic protein OP-1 has been described (see, e.g., Oppermann et al., U.S. Pat. No. 5,354,557, incorporated herein by reference). Natural-sourced osteogenic protein in its mature, native form is a glycosylated dimer typically having an apparent molecular weight of about 30-36 kDa as determined by SDS-PAGE. When reduced, the 30 kDa protein gives rise to two glycosylated peptide subunits having apparent molecular weights of about 16 kDa and 18 kDa. In the reduced state, the protein has no detectable osteogenic activity. The unglycosylated protein, which also has osteogenic activity, has an apparent molecular weight of about 27 kDa. When reduced, the 27 kDa protein gives rise to two unglycosylated polypeptides, having molecular weights of about 14 kDa to 16 kDa, capable of inducing endochondral bone formation in a mammal. Osteogenic proteins may include forms having varying glycosylation patterns, varying N-termini, and active truncated or mutated forms of native protein. As described above, particularly useful sequences include those comprising the C-terminal 96 or 102 amino acid sequences of DPP (from Drosophila), Vg1 (from Xenopus), Vgr-1 (from mouse), the OP-1 and OP-2 proteins, (see U.S. Pat. No. 5,011,691 and Oppermann et al., incorporated herein by reference), as well as the proteins referred to as BMP-2, BMP-3, BMP-4 (see WO88/00205, U.S. Pat. No. 5,013,649 and WO91/18098, incorporated herein by reference), BMP-5 and BMP-6 (see WO90/11366, PCT/US90/01630, incorporated herein by reference), BMP-8 and BMP-9.

Preferred osteogenic or morphogenic proteins of this invention comprise at least one polypeptide including, but not limited to OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, BMP-12, BMP-13, BMP-14, BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, CDMP-1, CDMP-2, CDMP-3, dorsalin-1, DPP, Vg-1, Vgr-1, 60A protein, NODAL, UNIVIN, SCREW, ADMP, NEURAL, amino acid sequence variants and homologs thereof, including species homologs, thereof and fragments thereof. In some embodiments, the protein comprises at least one polypeptide including, but not limited to OP-1 (BMP-7), OP-2, OP-3, BMP-2, BMP-3, BMP-3b, BMP-4, BMP-5, BMP-6, BMP-9, BMP-10, BMP-11, BMP-14, BMP-15, BMP-16, BMP-17, BMP-18, amino acid sequence variants and homologs thereof, including species homologs, thereof and fragments thereof. In other embodiments, the protein comprises at least one polypeptide including, but not limited to, GDF-1, GDF-2, GDF-3, GDF-5, GDF-6, GDF-7, CDMP-1, CDMP-2, CDMP-3, amino acid sequence variants and homologs thereof, including species homologs, thereof and fragments thereof. Preferably, the protein comprises at least one polypeptide selected from OP-1 (BMP-7), BMP-2, BMP-4, BMP-5, BMP-6, GDF-5, GDF-6, GDF-7, CDMP-1, CDMP-2 or CDMP-3; more preferably, OP-1 (BMP-7), BMP-5, BMP-6, GDF-5, GDF-6, GDF-7, CDMP-1, CDMP-2 or CDMP-3; even more preferably, OP-1 (BMP-7), BMP-5 or BMP-6; and most preferably, OP-1 (BMP-7).

Publications disclosing these sequences, as well as their chemical and physical properties, include: OP-1 and OP-2 (U.S. Pat. No. 5,011,691; U.S. Pat. No. 5,266,683; Ozkaynak et al., EMBO J., 9, pp. 2085-2093 (1990); OP-3 (WO94/10203 (PCT US93/10520)); BMP-2, BMP-3, BMP-4, (WO88/00205; Wozney et al. Science, 242, pp. 1528-1534 (1988)); BMP-5 and BMP-6, (Celeste et al., PNAS, 87, 9843-9847 (1991)); Vgr-1 (Lyons et al., PNAS, 86, pp. 4554-4558 (1989)); DPP (Padgett et al. Nature, 325, pp. 81-84 (1987)); Vg-1 (Weeks, Cell, 51, pp. 861-867 (1987)); BMP-9 (WO95/33830 (PCT/US95/07084); BMP-10 (WO94/26893 (PCT/US94/05290); BMP-11 (WO94/26892 (PCT/US94/05288); BMP-12 (WO95/16035 (PCT/US94/14030); BMP-13 (WO95/16035 (PCT/US94/14030); GDF-1 (WO92/00382 (PCT/US91/04096) and Lee et al. PNAS, 88, pp. 4250-4254 (1991); GDF-8 (WO94/21681 (PCT/US94/03019); GDF-9 (WO94/15966 (PCT/US94/00685); GDF-10 (WO95/10539 (PCT/US94/11440); GDF-11 (WO96/01845 (PCT/US95/08543); BMP-15 (WO96/36710 (PCT/US96/06540); MP-121 (WO96/01316 (PCT/EP95/02552); GDF-5 (CDMP-1, MP52) (WO94/15949 (PCT/US94/00657) and WO96/14335 (PCT/US94/12814) and WO93/16099 (PCT/EP93/00350)); GDF-6 (CDMP-2, BMPl3) (WO95/01801 (PCT/US94/07762) and WO96/14335 and WO95/10635 (PCT/US94/14030)); GDF-7 (CDMP-3, BMP12) (WO95/10802 (PCT/US94/07799) and WO95/10635 (PCT/US94/14030)); BMP-17 and BMP-18 (U.S. Pat. No. 6,027,917). The above publications are incorporated herein by reference.

In another embodiment, useful proteins include biologically active biosynthetic constructs, including novel biosynthetic bone morphogenetic proteins and chimeric proteins designed using sequences from two or more known bone morphogenetic proteins.

In one preferred embodiment of this invention, the bone morphogenetic protein comprises a pair of subunits disulfide bonded to produce a dimeric species, wherein at least one of the subunits comprises a recombinant peptide belonging to the BMP protein family. In another preferred embodiment of this invention, the bone morphogenetic protein comprises a pair of subunits that produce a dimeric species formed through non-covalent interactions, wherein at least one of the subunits comprises a recombinant peptide belonging to the BMP protein family. Non-covalent interactions include Van der Waals, hydrogen bond, hydrophobic and electrostatic interactions. The dimeric species may be a homodimer or heterodimer and is capable of inducing cell proliferation and/or tissue formation. In other preferred embodiments, the bone morphogenetic protein is a monomer.

In certain preferred embodiments, bone morphogenetic proteins useful herein include those in which the amino acid sequences comprise a sequence sharing at least 70% amino acid sequence homology or “similarity”, and preferably 75%, 80%, 85%, 90%, 95%, or 98% homology or similarity, with a reference bone morphogenetic protein selected from the foregoing naturally occurring proteins. Preferably, the reference protein is human OP-1, and the reference sequence thereof is the C-terminal seven cysteine domain present in osteogenically active forms of human OP-1, residues 330-431 of SEQ ID NO: 1. In certain embodiments, a polypeptide suspected of being functionally equivalent to a reference bone morphogenetic polypeptide is aligned therewith using the method of Needleman, et al., supra, implemented conveniently by computer programs such as the Align program (DNAstar, Inc.). As noted above, internal gaps and amino acid insertions in the candidate sequence are ignored for purposes of calculating the defined relationship, conventionally expressed as a level of amino acid sequence homology or identity, between the candidate and reference sequences.

“Amino acid sequence homology” is understood herein to include both amino acid sequence identity and similarity. Homologous sequences share identical and/or similar amino acid residues, where similar residues are conservation substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence. In a currently preferred embodiment, the reference sequence is OP-1. Bone morphogenetic proteins useful herein accordingly include allelic, phylogenetic counterpart and other variants of the preferred reference sequence, whether naturally-occurring or biosynthetically produced (e.g., including “muteins” or “mutant proteins”), as well as novel members of the general morphogenetic family of proteins, including those set forth and identified above. Certain particularly preferred bone morphogenetic polypeptides share at least 60% amino acid identity with the preferred reference sequence of human OP-1, still more preferably at least 65%, 70%, 75%, 80%, 85%, 90%, 95%, or 98% amino acid identity therewith.

In another embodiment, useful proteins include those sharing the conserved seven cysteine domain and sharing at least 70% amino acid sequence homology (similarity) within the C-terminal active domain, as defined herein.

In still another preferred embodiment, useful active proteins have polypeptide chains with amino acid sequences comprising a sequence encoded by a nucleic acid that hybridizes, under low, medium or high stringency hybridization conditions, to DNA or RNA encoding reference bone morphogenetic sequences, e.g., C-terminal sequences defining the conserved seven cysteine domains of OP-1, OP-2, BMP-2, BMP-4, BMP-5, BMP-6, 60A, GDF-3, GDF-5, GDF-6, GDF-7 and the like. As used herein, high stringent hybridization conditions are defined as hybridization according to known techniques in 40% formamide, 5×SSPE, 5×Denhardt's Solution, and 0.1% SDS at 37° C. overnight, and washing in 0.1×SSPE, 0.1% SDS at 50° C. Standard stringent conditions are well characterized in commercially available, standard molecular cloning texts. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984): Nucleic Acid Hybridization (B. D. Hames & S. J. Higgins eds. 1984); and B. Perbal, A Practical Guide To Molecular Cloning (1984), the disclosures of which are incorporated herein by reference.

As noted above, proteins useful in the present invention generally are dimeric proteins comprising a folded pair of the above polypeptides. Such bone morphogenetic proteins are inactive when reduced, but are active as oxidized homodimers and when oxidized in combination with others of this invention to produce heterodimers. Thus, members of a folded pair of bone morphogenetic polypeptides in a morphogenically active protein can be selected independently from any of the specific polypeptides mentioned above. In some embodiments, the bone morphogenetic protein is a monomer.

The bone morphogenetic proteins useful in the materials and methods of this invention include proteins comprising any of the polypeptide chains described above, whether isolated from naturally-occurring sources, or produced by recombinant DNA or other synthetic techniques, and includes allelic and phylogenetic counterpart variants of these proteins, as well as muteins thereof, fragments thereof and various truncated and fusion constructs. Deletion or addition mutants also are envisioned to be active, including those which may alter the conserved C-terminal six or seven cysteine domain, provided that the alteration does not functionally disrupt the relationship of these cysteines in the folded structure. Accordingly, such active forms are considered the equivalent of the specifically described constructs disclosed herein. The proteins may include forms having varying glycosylation patterns, varying N-termini, a family of related proteins having regions of amino acid sequence homology, and active truncated or mutated forms of native or biosynthetic proteins, produced by expression of recombinant DNA in host cells.

The bone morphogenetic proteins contemplated herein can be expressed from intact or truncated cDNA or from synthetic DNAs in prokaryotic or eukaryotic host cells, and purified, cleaved, refolded, and dimerized to form morphogenically active compositions. Currently preferred host cells include, without limitation, prokaryotes including E. coli or eukaryotes including yeast, or mammalian cells, such as CHO, COS or BSC cells. One of ordinary skill in the art will appreciate that other host cells can be used to advantage. Detailed descriptions of the bone morphogenetic proteins useful in the practice of this invention, including how to make, use and test them for osteogenic activity, are disclosed in numerous publications, including U.S. Pat. Nos. 5,266,683 and 5,011,691, the disclosures of which are incorporated by reference herein, as well as in any of the publications recited herein, the disclosures of which are incorporated herein by reference.

Thus, in view of this disclosure and the knowledge available in the art, skilled genetic engineers can isolate genes from cDNA or genomic libraries of various different biological species, which encode appropriate amino acid sequences, or construct DNAs from oligonucleotides, and then can express them in various types of host cells, including both prokaryotes and eukaryotes, to produce large quantities of active proteins capable of stimulating bone and cartilage morphogenesis in a mammal. In addition, the skilled worker can also prepare nucleic acid molecules which express the proteins of this invention in vivo.

In some embodiments, the bone morphogenetic protein includes, but is not limited to OP-1, OP-2, OP-3, BMP-2, BMP-3, BMP-4, BMP-5, BMP-6, BMP-8, BMP-9, BMP-10, BMP-11, BMP-12, BMP-13, BMP-15, BMP-16, BMP-17, BMP-18, DPP, Vg1, Vgr, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, NODAL, UNIVIN, SCREW, ADMP, NEURAL, and amino acid sequence variants thereof. In some embodiments, the bone morphogenetic protein comprises an amino acid sequence having at least 70% homology with the C-terminal 102-106 amino acids, including the conserved seven cysteine domain, of human OP-1, said bone morphogenetic protein being capable of inducing repair of bone and/or cartilage defects.

In a preferred embodiment, the bone morphogenetic protein is OP-1, BMP-5, BMP-6, GDF-5, GDF-6 and GDF-7, CDMP-1, CDMP-2 or CDMP-3. In a more preferred embodiment, the bone morphogenetic protein is OP-1, BMP-5 or BMP-6. In a most preferred embodiment, the bone morphogenetic protein is OP-1.

### Assay for Detecting Neutralizing Antibody to BMP

The invention provides for a method of detecting neutralizing antibodies to a bone morphogenetic protein (BMP) in a sample. The sample containing the potentially neutralizing antibodies is first contacted with a BMP prior to being incubated with a BMP-responsive cell that comprises at least one endogenous gene, the expression of which is capable of being modulated by exposure to said BMP. The expression of said endogenous gene is determined by isolating the mRNA from the BMP-responsive cell followed by preparing the cDNA corresponding to said mRNA using reverse transcription. The cDNA is then amplified by quantitative real-time polymerase chain reaction (QPCR). The amount of amplified endogenous gene is determined and compared to the amount of the same endogenous early gene amplified from control cells contacted with BMP alone. The presence of neutralizing antibodies in the sample is detected if the amount of amplified endogenous gene is less than or greater than the amount of the same endogenous gene amplified from control cells contacted with BMP alone. In some embodiments, the amount of amplified gene is greater than the amount of the same endogenous gene amplified from control cells. In other embodiments, the amount of amplified gene is less than the amount of the same endogenous gene amplified from control cells.

In some embodiments, the method of this invention further comprises the steps of amplifying a housekeeping gene from the cDNA of the sample-treated cells by quantitative real-time polymerase chain reaction (QPCR) and determining the amount of said housekeeping gene amplified. The determined amount of housekeeping gene is then used to normalize the determined amount of endogenous early gene. Normalizing gene expression to that of an endogenous gene allows to control for intra and inter-assay variations.

In some embodiments, the responses induced by positive and negative control samples are determined to ensure that the assay is functioning properly. Negative controls are typically serum samples from a subject that has not been exposed to the BMP. In some instances, the negative control samples may be pooled serum samples from untreated subjects. Negative control samples measure the biological response induced by the BMP alone. In some embodiments, a reading of biological activity is made from the BMP and cells prior to addition of the sample, serving as a integrated negative control.

In some embodiments, the control includes serum samples from subjects treated with a BMP. In other embodiments, the control includes serum samples from subjects treated with a BMP and further includes a positive neutralizing antibody. In some embodiments, the serum samples are pooled. In other embodiments, the serum is human serum.

Additional controls may include standard solutions containing a range of BMP concentrations. These standards are individually incubated with the cells in the absence of a test sample, thereby generating a profile of biological responses induced at varying BMP concentrations.

Comparison of the response induced by the test sample with those induced by the standards allows for quantitation of the neutralizing activity contained in the test sample. For example, a test sample that induced a response equal to that of a standard containing 50% of the BMP concentration used in the test sample has neutralized 50% of the BMP activity. Units of neutralizing activity may be determined for each test sample using these comparisons.

In some embodiments, the sample containing the potentially neutralizing antibodies is first contacted with a BMP. The amount of BMP used in the assay is an amount sufficient to induce the biological response in the cell. However, detection of neutralizing antibodies is most sensitive when the amount of BMP used is not in excess of the amount sufficient to induce the maximum biological response in the cell. BMPs in excess of this amount may bind to neutralizing antibodies within a sample without a detectable change in the assay readout.

The amount of BMP will vary with the parameters of the assay, such as the amount of cells used, the size of the assay vessel and the sensitivity of the cells to the BMP. The amount of BMP can be titrated to determine the optimal amount (or the “effective concentration”) sufficient to induce the biological response in the cell without saturating the assay with excess BMP.

In some embodiments, the concentration of BMP used in the assay may be the following: at least 1 pg/mL, at least 100 pg/mL, at least 500 pg/mL, at least 1 ng/mL, at least 10 ng/mL, at least 15 ng/mL, at least 20 ng/mL, at least 25 ng/mL, at least 30 ng/mL, at least 40 ng/mL, at least 50 ng/mL, at least 60 ng/mL, at least 70 ng/mL, at least 80 ng/mL, at least 90 ng/mL, at least 100 ng/mL, at least 200 ng/mL, at least 500 ng/mL, at least 1 μg/mL, at least 100 μg/mL, at least 500 μg/mL, or at least 1 mg/mL.

In some embodiments, the samples of this invention may be any bodily fluid capable of containing neutralizing antibodies or proteins. Examples include, but are not limited to, blood, serum, lymph, plasma, synovial fluid, cerebrospinal fluid, lachrymal fluid, biopsy or tissue sample, cell suspension, saliva, oral fluid, mucus, amniotic fluid, colostrums, mammary gland secretions, urine, sweat and tissue culture medium.

In some embodiments, the samples of this invention may be assayed at multiple dilutions to obtain an accurate quantitation of neutralizing activity present in the sample. The units of neutralizing activity in each sample may be calculated based on the amount each sample was diluted.

In some embodiments, the samples of this invention may also be diluted to avoid interference from non-specific background components of the samples. For example, proteins found at high concentrations in the serum may, in some circumstances, non-specifically interact with components of the assay and reduce the sensitivity of the assay. Sample dilution may reduce or eliminate non-specific binding and thereby increase the signal-to-noise ratio of the assay.

In some embodiments, the samples of this invention may be assayed undiluted. In other embodiments, the samples of this invention may be assayed at dilution factors such as, for example, 1:1, 1:2, 1:5, 1:10, 1:15, 1:20, 1:30, 1:40, 1:50, 1:60, 1:80, 1:100, 1:500, 1:1000, or 1:5000. In other embodiments, the samples of the invention may be assayed at still greater dilution factors.

In some embodiments, the BMP-responsive cells of this invention may be any population of cells that expresses BMP receptors and allows the transduction of BMP-induced protein signaling, resulting in the activation of gene expression regulatory elements in response to the interaction between BMP and the cell receptors. In some embodiments, the assays of this invention may use one cell or a population of cells. In some embodiments, the BMP-responsive cell includes, but is not limited to, A549, SAOS-2, ROS and CSC12. In other embodiments, the BMP-responsive cell is a A549 cell. In some embodiments, the BMP-responsive cell includes, but is not limited to, primary stromal cells derived from either bone marrow, fat or muscle.

Cells are grown at any density appropriate for normal cell growth when used in the assays of this invention. The number of cells used to achieve an appropriate density is determined in part by the size and surface area of the tissue culture dish or plate used in the assay.

Cells may be used in the assay at any density. In some embodiments, the cells may be used in the assays at the following cell densities: at least 10% confluent, at least 25% confluent, at least 50% confluent, at least 80% confluent, at least 90% confluent, or at least 99% confluent.

In some embodiments, the cells are used in the assays of this invention at the following concentrations at least 1×10³/mL, at least 5×10³/mL, at least 1×10⁴/mL, at least 5×10⁴/mL, at least 1×10⁵/mL, at least 5×10⁵/mL, at least 1×10⁶/mL, at least 5×10⁶/mL or at least 1×10⁷/mL. In some embodiments, 1 cell/sample to 10⁹ cells/sample may be used in the assays of this invention.

In some embodiments, the cells are cell lines. In some embodiments, the cells are cells isolated from a subject and cultured ex vivo.

In some embodiments, the cells of this invention may be mammalian or non-mammalian. The cells may be of any species, including, but not limited to, human, monkey, mouse, rat, hamster and other vertebrate species. The cells may also be from invertebrate species such as, for example, insect cells. The cells may also be prokaryotic cells such as, for example, bacterial cells.

Cells may be cultured according to any of techniques known in the art. Cells may be grown in any culture flask, plate or dish suitable for cell culture. Media, supplements and culture conditions appropriate for cell culture are well known to those of skill in the art.

In some embodiments, the methods of this invention measures an endogenous early gene, the expression of which is capable of being modulated by exposure to BMPs. Monitoring endogenous gene transcription is more reflective of in vivo activity than measuring expression from a recombinant construct containing a short promoter region driving a reporter gene. Furthermore, measuring gene expression early on following BMP treatment allows for higher assay specificity and is less prone to interference from non BMP components found in the serum samples. For example, a clear correlation between cell toxicity induced by a number of human serum samples and low signal measurements in a BMP response element driven luciferase reporter assay. In contrast, the QPCR assay read out was not affected by the viability (toxicity) of the cell.

In some embodiments, the endogenous gene, the expression of which is capable of being modulated by exposure to a BMP includes, but is not limited to, a gene selected from a gene set forth in Table 1:

TABLE 1

| Gene Title | Gene Symbol |
|---|---|
| 4-aminobutyrate | ABAT |
| aminotransferase | |
| ATP-binding cassette, sub- | ABCA1 |
| family A (ABC1), member 1 | |
| actin, alpha, cardiac muscle | ACTC |
| a disintegrin-like and | ADAMTS9 |
| metalloprotease (reprolysin | |
| type) with thrombospondin | |
| typ | |
| a disintegrin-like and | ADAMTS9 |
| metalloprotease (reprolysin | |
| type) with thrombospondin | |
| typ | |
| ADAMTS-like 3 | ADAMTSL3 |
| AF15q14 protein | AF15Q14 |
| ALL1-fused gene from | AF1Q |
| chromosome 1q /// ALL1- | |
| fused gene from | |
| chromosome 1q | |
| aggrecan 1 (chondroitin | AGC1 |
| sulfate proteoglycan 1, large | |
| aggregating proteoglycan, | |
| aggrecan 1 (chondroitin | AGC1 |
| sulfate proteoglycan 1, large | |
| aggregating proteoglycan, | |
| aggrecan 1 (chondroitin | AGC1 |
| sulfate proteoglycan 1, large | |
| aggregating proteoglycan, | |
| A kinase (PRKA) anchor | AKAP12 |
| protein (gravin) 12 | |
| A kinase (PRKA) anchor | AKAP12 |
| protein (gravin) 12 | |
| A kinase (PRKA) anchor | AKAP12 |
| protein (gravin) 12 | |
| A kinase (PRKA) anchor | AKAP7 |
| protein 7 | |
| alkaline phosphatase, | ALPL |
| liver/bone/kidney | |
| adenosylmethionine | AMD1 |
| decarboxylase 1 | |
| adenosylmethionine | AMD1 |
| decarboxylase 1 | |
| amphoterin induced gene 2 | AMIGO2 |
| angiopoietin-like 2 | ANGPTL2 |
| angiopoietin-like 2 | ANGPTL2 |
| angiopoietin-like 2 | ANGPTL2 |
| anillin, actin binding protein | ANLN |
| (scraps homolog, | |
| Drosophila) | |
| anillin, actin binding protein | ANLN |
| (scraps homolog, | |
| Drosophila) | |
| anthrax toxin receptor 1 | ANTXR1 |
| anthrax toxin receptor 1 | ANTXR1 |
| apolipoprotein B mRNA | APOBEC3B |
| editing enzyme, catalytic | |
| polypeptide-like 3B | |
| Rho GTPase activating | ARHGAP21 |
| protein 21 | |
| ADP-ribosylation factor-like 7 | ARL7 |
| ADP-ribosylation factor-like 7 | ARL7 |
| asporin (LRR class 1) | ASPN |
| ATPase family, AAA domain | ATAD2 |
| containing 2 | |
| Atonal homolog 8 | ATOH8 |
| (Drosophila) | |
| atonal homolog 8 | ATOH8 |
| (Drosophila) | |
| ATPase, Na+/K+ | ATP1B1 |
| transporting, beta 1 | |
| polypeptide | |
| aurora kinase B | AURKB |
| BMP and activin membrane- | BAMBI |
| bound inhibitor homolog | |
| (Xenopus laevis) | |
| basic helix-loop-helix | BHLHB2 |
| domain containing, class B, 2 | |
| baculoviral IAP repeat- | BIRC5 |
| containing 5 (survivin) | |
| baculoviral IAP repeat- | BIRC5 |
| containing 5 (survivin) | |
| baculoviral IAP repeat- | BIRC5 |
| containing 5 (survivin) | |
| Basonuclin 2 | BNC2 |
| breast cancer 1, early onset | BRCA1 |
| BTB (POZ) domain | BTBD14A |
| containing 14A | |
| basic transcription element | BTEB1 |
| binding protein 1 | |
| BUB1 budding uninhibited | BUB1B |
| by benzimidazoles 1 | |
| homolog beta (yeast) | |
| chromosome 10 open | C10orf3 |
| reading frame 3 | |
| chromosome 18 open | C18orf1 |
| reading frame 1 | |
| chromosome 18 open | C18orf24 |
| reading frame 24 | |
| C1q and tumor necrosis | C1QTNF1 |
| factor related protein 1 /// | |
| C1q and tumor necrosis | |
| facto | |
| C1q and tumor necrosis | C1QTNF5 |
| factor related protein 5 | |
| chromosome 20 open | C20orf129 |
| reading frame 129 | |
| chromosome 20 open | C20orf39 |
| reading frame 39 | |
| chromosome 20 open | C20orf82 |
| reading frame 82 | |
| chromosome 22 open | C22orf18 |
| reading frame 18 | |
| chromosome 2 open reading | C2orf31 |
| frame 31 /// chromosome 2 | |
| open reading frame 31 | |
| calcitonin-related | CALCB |
| polypeptide, beta | |
| caspase recruitment domain | CARD10 |
| family, member 10 | |
| chemokine (C-C motif) | CCL2 |
| ligand 2 | |
| cyclin B1 | CCNB1 |
| cyclin B1 | CCNB1 |
| cyclin B2 | CCNB2 |
| cyclin E2 | CCNE2 |
| CD24 antigen (small cell | CD24 |
| lung carcinoma cluster 4 | |
| antigen) | |
| CD24 antigen (small cell | CD24 |
| lung carcinoma cluster 4 | |
| antigen) | |
| CD24 antigen (small cell | CD24 |
| lung carcinoma cluster 4 | |
| antigen) | |
| CD24 antigen (small cell | CD24 |
| lung carcinoma cluster 4 | |
| antigen) | |
| CD24 antigen (small cell | CD24 |
| lung carcinoma cluster 4 | |
| antigen) | |
| cell division cycle 2, G1 to S | CDC2 |
| and G2 to M | |
| cell division cycle 2, G1 to S | CDC2 |
| and G2 to M | |
| CDC20 cell division cycle 20 | CDC20 |
| homolog (S. cerevisiae) | |
| CDC6 cell division cycle 6 | CDC6 |
| homolog (S. cerevisiae) | |
| CDC6 cell division cycle 6 | CDC6 |
| homolog (S. cerevisiae) | |
| cell division cycle associated 1 | CDCA1 |
| cell division cycle associated 3 | CDCA3 |
| cyclin-dependent kinase | CDKN1C |
| inhibitor 1C (p57, Kip2) | |
| cyclin-dependent kinase | CDKN3 |
| inhibitor 3 (CDK2-associated | |
| dual specificity phosphatas | |
| cyclin-dependent kinase | CDKN3 |
| inhibitor 3 (CDK2-associated | |
| dual specificity phosphatas | |
| CCAAT/enhancer binding | CEBPA |
| protein (C/EBP), alpha | |
| centromere protein A, 17 kDa | CENPA |
| centromere protein F, | CENPF |
| 350/400ka (mitosin) | |
| creatine kinase, brain | CKB |
| chloride intracellular channel 3 | CLIC3 |
| collagen, type XI, alpha 1 | COL11A1 |
| Collagen, type XIV, alpha 1 | COL14A1 |
| (undulin) | |
| collagen, type IX, alpha 2 | COL9A2 |
| cartilage oligomeric matrix | COMP |
| protein | |
| core promoter element | COPEB |
| binding protein | |
| copine V | CPNE5 |
| cytokine receptor-like factor 1 | CRLF1 |
| crystallin, alpha B | CRYAB |
| cysteine and glycine-rich | CSRP2 |
| protein 2 | |
| cysteine and glycine-rich | CSRP2 |
| protein 2 | |
| chondroitin sulfate synthase 3 | CSS3 |
| cystatin A (stefin A) | CSTA |
| CTP synthase | CTPS |
| cathepsin C | CTSC |
| cytoglobin | CYGB |
| dachsous 1 (Drosophila) | DCHS1 |
| DNA-damage-inducible | DDIT4L |
| transcript 4-like | |
| DEP domain containing 1 | DEPDC1 |
| DEP domain containing 1 | DEPDC1 |
| DEP domain containing 1B | DEPDC1B |
| deiodinase, iodothyronine, | DIO2 |
| type II | |
| deiodinase, iodothyronine, | DIO2 |
| type II | |
| hypothetical protein | DKFZP434B044 |
| DKFZp434B044 | |
| hypothetical protein | DKFZP761M1511 |
| DKFZP761M1511 | |
| Hypothetical protein | DKFZp762A217 |
| DKFZp762A217 | |
| hypothetical protein | DKFZp762A217 |
| DKFZp762A217 | |
| dickkopf homolog 1 | DKK1 |
| (Xenopus laevis) | |
| discs, large homolog 7 | DLG7 |
| (Drosophila) | |
| distal-less homeo box 1 | DLX1 |
| distal-less homeo box 2 | DLX2 |
| distal-less homeo box 3 | DLX3 |
| distal-less homeo box 5 | DLX5 |
| Distal-less homeo box 6 | DLX6 |
| Distal-less homeo box 6 | DLX6 |
| dynein, cytoplasmic, | DNCI1 |
| intermediate polypeptide 1 | |
| dual specificity phosphatase 2 | DUSP2 |
| dual specificity phosphatase 4 | DUSP4 |
| dual specificity phosphatase 6 | DUSP6 |
| dysferlin, limb girdle | DYSF |
| muscular dystrophy 2B | |
| (autosomal recessive) | |
| extracellular matrix protein | ECM2 |
| 2, female organ and | |
| adipocyte specific | |
| endothelial differentiation, | EDG3 |
| sphingolipid G-protein- | |
| coupled receptor, 3 | |
| endothelin 1 | EDN1 |
| endothelin receptor type A | EDNRA |
| endothelin receptor type A | EDNRA |
| Endothelin receptor type A | EDNRA |
| EF hand domain containing 1 | EFHD1 |
| early growth response 3 | EGR3 |
| eukaryotic translation | EIF2B2 |
| initiation factor 2B, subunit 2 | |
| beta, 39 kDa | |
| elastin (supravalvular aortic | ELN |
| stenosis, Williams-Beuren | |
| syndrome) | |
| ectodermal-neural cortex | ENC1 |
| (with BTB-like domain) | |
| ectodermal-neural cortex | ENC1 |
| (with BTB-like domain) | |
| coagulation factor III | F3 |
| (thromboplastin, tissue | |
| factor) | |
| fatty acid binding protein 5 | FABP5 |
| (psoriasis-associated) | |
| fibulin 2 | FBLN2 |
| F-box protein 5 | FBXO5 |
| flap structure-specific | FEN1 |
| endonuclease 1 | |
| fibroblast growth factor 5 | FGF5 |
| fibroblast growth factor 7 | FGF7 /// |
| (keratinocyte growth factor) | FLJ30435 |
| /// hypothetical protein | |
| fibroblast growth factor | FGFR3 |
| receptor 3 (achondroplasia, | |
| thanatophoric dwarfism) | |
| fibroblast growth factor | FGFR3 |
| receptor 3 (achondroplasia, | |
| thanatophoric dwarfism) | |
| fidgetin-like 1 | FIGNL1 |
| hypothetical protein | FLJ10156 |
| FLJ10156 | |
| hypothetical protein | FLJ10520 |
| FLJ10520 | |
| hypothetical protein | FLJ10719 |
| FLJ10719 | |
| hypothetical protein | FLJ20105 |
| FLJ20105 | |
| hypothetical protein | FLJ20701 |
| FLJ20701 /// hypothetical | |
| protein FLJ20701 | |
| hypothetical protein | FLJ23191 |
| FLJ23191 | |
| FLJ23311 protein | FLJ23311 |
| hypothetical protein | FLJ40629 |
| FLJ40629 | |
| forkhead box M1 | FOXM1 |
| Fraser syndrome 1 | FRAS1 |
| frizzled-related protein | FRZB |
| FtsJ homolog 1 (E. coli) | FTSJ1 |
| frizzled homolog 1 | FZD1 |
| (Drosophila) | |
| frizzled homolog 8 | FZD8 |
| (Drosophila) /// frizzled | |
| homolog 8 (Drosophila) | |
| frizzled homolog 8 | FZD8 |
| (Drosophila) | |
| GABA(A) receptor- | GABARAPL1 |
| associated protein like 1 | |
| GABA(A) receptor- | GABARAPL1 |
| associated protein like 1 | |
| growth differentiation factor | GDF5 |
| 5 (cartilage-derived | |
| morphogenetic protein-1) | |
| growth hormone receptor | GHR |
| guanine nucleotide binding | GNAI1 |
| protein (G protein), alpha | |
| inhibiting activity polype | |
| guanine nucleotide binding | GNG2 |
| protein (G protein), gamma 2 | |
| Golgi associated PDZ and | GOPC |
| coiled-coil motif containing | |
| glycerol-3-phosphate | GPD1L |
| dehydrogenase 1-like | |
| gremlin 1 homolog, cysteine | GREM1 |
| knot superfamily (Xenopus | |
| laevis) | |
| gremlin 2 homolog, cysteine | GREM2 |
| knot superfamily (Xenopus | |
| laevis) | |
| gremlin 2 homolog, cysteine | GREM2 |
| knot superfamily (Xenopus | |
| laevis) | |
| G protein-coupled receptor | GRK5 |
| kinase 5 | |
| G-2 and S-phase expressed 1 | GTSE1 |
| glycogenin 2 | GYG2 |
| glycogenin 2 | GYG2 |
| hyaluronan and | HAPLN3 |
| proteoglycan link protein 3 | |
| hyaluronan synthase 2 | HAS2 |
| hyaluronan synthase 3 | HAS3 |
| chromosome condensation | HCAP-G |
| protein G | |
| chromosome condensation | HCAP-G |
| protein G | |
| Helicase, lymphoid-specific | HELLS |
| Helicase, lymphoid-specific | HELLS |
| hairy/enhancer-of-split | HEY1 |
| related with YRPW motif 1 | |
| hairy/enhancer-of-split | HEY1 |
| related with YRPW motif 1 | |
| hyaluronan-mediated motility | HMMR |
| receptor (RHAMM) | |
| hyaluronan-mediated motility | HMMR |
| receptor (RHAMM) | |
| histamine N- | HNMT |
| methyltransferase | |
| heterogeneous nuclear | HNRPDL |
| ribonucleoprotein D-like | |
| heat shock 70 kDa protein 2 | HSPA2 |
| HSPC150 protein similar to | HSPC150 |
| ubiquitin-conjugating | |
| enzyme | |
| inhibitor of DNA binding 1, | ID1 |
| dominant negative helix- | |
| loop-helix protein | |
| inhibitor of DNA binding 2, | ID2 |
| dominant negative helix- | |
| loop-helix protein | |
| inhibitor of DNA binding 3, | ID3 |
| dominant negative helix- | |
| loop-helix protein | |
| inhibitor of DNA binding 4, | ID4 |
| dominant negative helix- | |
| loop-helix protein | |
| Inhibitor of DNA binding 4, | ID4 |
| dominant negative helix- | |
| loop-helix protein | |
| insulin-like growth factor 1 | IGF1 |
| (somatomedin C) | |
| insulin-like growth factor 1 | IGF1 |
| (somatomedin C) | |
| insulin-like growth factor | IGFBP5 |
| binding protein 5 | |
| insulin-like growth factor | IGFBP5 |
| binding protein 5 | |
| insulin-like growth factor | IGFBP5 |
| binding protein 5 | |
| Insulin-like growth factor | IGFBP7 |
| binding protein 7 | |
| interleukin 6 (interferon, beta | IL6 |
| 2) | |
| interleukin 7 receptor /// | IL7R |
| interleukin 7 receptor | |
| Interleukin 7 receptor | IL7R |
| interleukin 8 | IL8 |
| inhibin, beta A (activin A, | INHBA |
| activin AB alpha | |
| polypeptide) | |
| Inhibin, beta A (activin A, | INHBA |
| activin AB alpha | |
| polypeptide) | |
| Integrin, alpha 9 | ITGA9 |
| influenza virus NS1A binding | IVNS1ABP |
| protein | |
| jagged 1 (Alagille syndrome) | JAG1 |
| jagged 1 (Alagille syndrome) | JAG1 |
| potassium voltage-gated | KCNE4 |
| channel, lsk-related family, | |
| member 4 | |
| potassium channel, | KCNK2 |
| subfamily K, member 2 | |
| potassium channel | KCTD15 |
| tetramerisation domain | |
| containing 15 | |
| potassium channel | KCTD15 |
| tetramerisation domain | |
| containing 15 | |
| KIAA0186 gene product | KIAA0186 |
| KIAA0527 protein | KIAA0527 |
| KIAA1026 protein | KIAA1026 |
| KIAA1102 protein | KIAA1102 |
| KIAA1102 protein | KIAA1102 |
| KIAA1102 protein | KIAA1102 |
| KIAA1912 protein | KIAA1912 |
| kinesin family member 11 | KIF11 |
| Kinesin family member 14 | KIF14 |
| kinesin family member 20A | KIF20A |
| kinesin family member 23 | KIF23 |
| kinesin family member 2C | KIF2C |
| kinesin family member 4A | KIF4A |
| v-kit Hardy-Zuckerman 4 | KIT |
| feline sarcoma viral | |
| oncogene homolog | |
| kinesin-like 7 | KNSL7 |
| karyopherin alpha 2 (RAG | KPNA2 |
| cohort 1, importin alpha 1) /// | |
| karyopherin alpha 2 (RA | |
| keratin 7 | KRT7 |
| keratin associated protein 1- | KRTAP1-3 |
| 3 /// keratin associated | |
| protein 1-3 | |
| Keratin associated protein 2-4 | KRTAP2-4 |
| likely ortholog of mouse | LBH |
| limb-bud and heart gene /// | |
| likely ortholog of mouse li | |
| LIM domain binding 3 | LDB3 |
| lymphoid enhancer-binding | LEF1 |
| factor 1 | |
| lunatic fringe homolog | LFNG |
| (Drosophila) | |
| legumain | LGMN |
| Luteinizing | LHCGR |
| hormone/choriogonadotropin | |
| receptor | |
| LIM domain kinase 2 | LIMK2 |
| lin-7 homolog B (C. elegans) | LIN7B |
| lin-7 homolog B (C. elegans) | LIN7B |
| lamin B1 | LMNB1 |
| LIM domain only 2 | LMO2 |
| (rhombotin-like 1) | |
| hypothetical protein | LOC146909 |
| LOC146909 | |
| hypothetical protein | LOC150837 |
| LOC150837 | |
| hypothetical protein | LOC157570 |
| LOC157570 | |
| hypothetical protein | LOC163782 |
| LOC163782 | |
| similar to ribosomal protein | LOC200916 |
| L22 | |
| hypothetical protein | LOC201725 |
| LOC201725 | |
| secretory protein | LOC284013 |
| LOC284013 | |
| hypothetical protein | LOC285705 |
| LOC285705 | |
| hypothetical protein | LOC285758 |
| LOC285758 | |
| hypothetical protein | LOC339924 |
| LOC339924 | |
| Hypothetical LOC387763 | LOC387763 |
| Tetraspanin similiar to | LOC90139 |
| uroplakin 1 | |
| hypothetical protein | LOC92270 |
| LOC92270 | |
| leucine-rich repeats and | LRIG1 |
| immunoglobulin-like | |
| domains 1 /// leucine-rich | |
| repeats | |
| low density lipoprotein | LRP4 |
| receptor-related protein 4 | |
| leucine rich repeat | LRRC15 |
| containing 15 | |
| MAD2 mitotic arrest | MAD2L1 |
| deficient-like 1 (yeast) | |
| MAD2 mitotic arrest | MAD2L1 |
| deficient-like 1 (yeast) | |
| Mitogen-activated protein | MAP3K4 |
| kinase kinase kinase 4 | |
| melanoma cell adhesion | MCAM |
| molecule | |
| melanoma cell adhesion | MCAM |
| molecule | |
| melanoma cell adhesion | MCAM |
| molecule | |
| melanoma cell adhesion | MCAM |
| molecule | |
| MCM10 minichromosome | MCM10 |
| maintenance deficient 10 (S. cerevisiae) | |
| MCM10 minichromosome | MCM10 |
| maintenance deficient 10 (S. cerevisiae) | |
| MCM10 minichromosome | MCM10 |
| maintenance deficient 10 (S. cerevisiae) | |
| MCM8 minichromosome | MCM8 |
| maintenance deficient 8 (S. cerevisiae) | |
| maternal embryonic leucine | MELK |
| zipper kinase | |
| mesenchyme homeo box 2 | MEOX2 |
| (growth arrest-specific | |
| homeo box) | |
| mesoderm specific transcript | MEST |
| homolog (mouse) | |
| milk fat globule-EGF factor 8 | MFGE8 |
| protein | |
| antigen p97 (melanoma | MFI2 |
| associated) identified by | |
| monoclonal antibodies 133.2 | |
| and | |
| Hypothetical protein | MGC13057 |
| MGC13057 | |
| hypothetical protein | MGC24103 |
| MGC24103 | |
| tubulin, beta polypeptide | MGC8685 |
| paralog | |
| monoglyceride lipase /// | MGLL |
| monoglyceride lipase | |
| monoglyceride lipase | MGLL |
| matrix Gla protein | MGP |
| antigen identified by | MKI67 |
| monoclonal antibody Ki-67 | |
| antigen identified by | MKI67 |
| monoclonal antibody Ki-67 | |
| MLF1 interacting protein | MLF1IP |
| meningioma (disrupted in | MN1 |
| balanced translocation) 1 | |
| Mitochondrial ribosomal | MRPS6 |
| protein S6 | |
| msh homeo box homolog 1 | MSX1 |
| (Drosophila) | |
| msh homeo box homolog 2 | MSX2 |
| (Drosophila) | |
| more than blood homolog | MTB |
| V-myb myeloblastosis viral | MYBL1 |
| oncogene homolog (avian)- | |
| like 1 | |
| NEDD4-related E3 ubiquitin | NEDL2 |
| ligase NEDL2 | |
| NIMA (never in mitosis gene | NEK2 |
| a)-related kinase 2 | |
| neogenin homolog 1 | NEO1 |
| (chicken) | |
| neuropilin (NRP) and tolloid | NETO2 |
| (TLL)-like 2 | |
| nicotinamide N- | NNMT |
| methyltransferase | |
| noggin | NOG |
| nuclear receptor subfamily | NR4A2 |
| 4, group A, member 2 | |
| neurotrophic tyrosine kinase, | NTRK2 |
| receptor, type 2 | |
| nucleolar and spindle | NUSAP1 |
| associated protein 1 | |
| nucleolar and spindle | NUSAP1 |
| associated protein 1 | |
| Opa-interacting protein 5 | OIP5 |
| olfactomedin-like 2B | OLFML2B |
| PTPL1-associated RhoGAP 1 | PARG1 |
| protocadherin beta 10 | PCDHB10 |
| protocadherin beta 14 | PCDHB14 |
| protocadherin beta 7 | PCDHB7 |
| DNA replication complex | Pfs2 |
| GINS protein PSF2 | |
| placental growth factor, | PGF |
| vascular endothelial growth | |
| factor-related protein | |
| placenta-specific 8 | PLAC8 |
| plasminogen activator, | PLAT |
| tissue | |
| pleckstrin homology domain | PLEKHH2 |
| containing, family H (with | |
| MyTH4 domain) member 2 | |
| polo-like kinase 4 | PLK4 |
| (Drosophila) | |
| plexin A2 | PLXNA2 |
| podocalyxin-like | PODXL |
| polymerase (DNA directed), | POLE2 |
| epsilon 2 (p59 subunit) | |
| polymerase (DNA directed), | POLE3 |
| epsilon 3 (p17 subunit) | |
| PTPRF interacting protein, | PPFIBP2 |
| binding protein 2 (liprin beta | |
| 2) | |
| peptidylprolyl isomerase F | PPIF |
| (cyclophilin F) | |
| protein phosphatase 1, | PPP1R3C |
| regulatory (inhibitor) subunit | |
| 3C | |
| protein regulator of | PRC1 |
| cytokinesis 1 | |
| proline arginine-rich end | PRELP |
| leucine-rich repeat protein | |
| primase, polypeptide 2A, | PRIM2A |
| 58 kDa | |
| paired related homeobox 2 | PRRX2 |
| protease, serine, 35 | PRSS35 |
| phosphoribosyl transferase | PRTFDC1 |
| domain containing 1 | |
| prostaglandin-endoperoxide | PTGS2 |
| synthase 2 (prostaglandin | |
| G/H synthase and | |
| cyclooxyge | |
| prostaglandin-endoperoxide | PTGS2 |
| synthase 2 (prostaglandin | |
| G/H synthase and | |
| cyclooxyge | |
| protein tyrosine | PTPLB |
| phosphatase-like (proline | |
| instead of catalytic arginine), | |
| membe | |
| Rac GTPase activating | RACGAP1 |
| protein 1 | |
| RAD51 associated protein 1 | RAD51AP1 |
| RA-regulated nuclear matrix- | RAMP |
| associated protein | |
| RA-regulated nuclear matrix- | RAMP |
| associated protein | |
| retinoblastoma-like 1 (p107) | RBL1 |
| RNA binding motif protein 24 | RBM24 |
| replication factor C (activator | RFC3 |
| 1) 3, 38 kDa | |
| ral guanine nucleotide | RGL1 |
| dissociation stimulator-like 1 | |
| regulator of G-protein | RGS3 |
| signalling 3 | |
| regulator of G-protein | RGS4 |
| signalling 4 | |
| rhomboid, veinlet-like 2 | RHBDL2 |
| (Drosophila) | |
| Ras-induced senescence 1 | RIS1 |
| ribonucleotide reductase M1 | RRM1 |
| polypeptide | |
| ribonucleotide reductase M2 | RRM2 |
| polypeptide | |
| ribonucleotide reductase M2 | RRM2 |
| polypeptide | |
| stoned B/TFIIA-alpha/beta- | SALF /// ALF |
| like factor /// TFIIA- | |
| alpha/beta-like factor | |
| sterile alpha motif domain | SAMD11 |
| containing 11 | |
| scinderin | SCIN |
| CTCL tumor antigen se57-1 | SE57-1 |
| sema domain, | SEMA6D |
| transmembrane domain | |
| (TM), and cytoplasmic | |
| domain, (semaphorin) 6D | |
| sema domain, | SEMA6D |
| transmembrane domain | |
| (TM), and cytoplasmic | |
| domain, (semaphorin) 6D | |
| selenoprotein P, plasma, 1 | SEPP1 |
| serine (or cysteine) | SERPINB2 |
| proteinase inhibitor, clade B | |
| (ovalbumin), member 2 | |
| serine (or cysteine) | SERPINE1 |
| proteinase inhibitor, clade E | |
| (nexin, plasminogen | |
| activator | |
| serine (or cysteine) | SERPINE1 |
| proteinase inhibitor, clade E | |
| (nexin, plasminogen | |
| activator | |
| Sestrin 3 | SESN3 |
| shugoshin-like 2 (S. pombe) | SGOL2 |
| SH2-B homolog | SH2B |
| SHC SH2-domain binding | SHCBP1 |
| protein 1 | |
| solute carrier family 27 (fatty | SLC27A3 |
| acid transporter), member 3 | |
| solute carrier family 40 (iron- | SLC40A1 |
| regulated transporter), | |
| member 1 | |
| SMAD, mothers against | SMAD6 |
| DPP homolog 6 (Drosophila) | |
| SMAD, mothers against | SMAD7 |
| DPP homolog 7 (Drosophila) | |
| SPARC related modular | SMOC1 |
| calcium binding 1 | |
| snail homolog 1 (Drosophila) | SNAI1 |
| synuclein, alpha interacting | SNCAIP |
| protein (synphilin) | |
| suppressor of cytokine | SOCS2 |
| signaling 2 | |
| sclerosteosis | SOST |
| SRY (sex determining region | SOX4 |
| Y)-box 4 | |
| SRY (sex determining region | SOX4 |
| Y)-box 4 | |
| SRY (sex determining region | SOX4 |
| Y)-box 4 | |
| SRY (sex determining region | SOX4 |
| Y)-box 4 | |
| SRY (sex determining region | SOX9 |
| Y)-box 9 (campomelic | |
| dysplasia, autosomal sex- | |
| revers | |
| Sp7 transcription factor | SP7 |
| sperm associated antigen 5 | SPAG5 |
| kinetochore protein Spc25 | Spc25 |
| Suppression of | ST7L |
| tumorigenicity 7 like | |
| signal transducer and | STAT4 |
| activator of transcription 4 | |
| Stanniocalcin 2 | STC2 |
| stanniocalcin 2 | STC2 |
| serine/threonine kinase 6 | STK6 |
| serine/threonine kinase 6 | STK6 |
| sulfatase 2 | SULF2 |
| Synaptopodin 2 | SYNPO2 |
| Synaptopodin 2 | SYNPO2 |
| synaptopodin 2 | SYNPO2 |
| synaptopodin 2 | SYNPO2 |
| synaptotagmin XV | SYT15 |
| transforming, acidic coiled- | TACC3 |
| coil containing protein 3 | |
| TAFA protein 5 | TAFA5 |
| TAFA protein 5 | TAFA5 |
| TPR domain, ankyrin-repeat | TANC |
| and coiled-coil-containing | |
| T-box 2 | TBX2 |
| T-box 3 (ulnar mammary | TBX3 |
| syndrome) | |
| transforming growth factor, | TGFB3 |
| beta 3 | |
| thrombospondin 1 | THBS1 |
| thymidine kinase 1, soluble | TK1 |
| thymidine kinase 1, soluble | TK1 |
| transmembrane 4 | TM4SF1 |
| superfamily member 1 | |
| transmembrane 4 | TM4SF1 |
| superfamily member 1 | |
| tumor necrosis factor | TNFRSF11B |
| receptor superfamily, | |
| member 11b | |
| (osteoprotegerin) | |
| tumor necrosis factor | TNFRSF11B |
| receptor superfamily, | |
| member 11b | |
| (osteoprotegerin) | |
| topoisomerase (DNA) II | TOP2A |
| alpha 170 kDa | |
| topoisomerase (DNA) II | TOP2A |
| alpha 170 kDa | |
| T-LAK cell-originated protein | TOPK |
| kinase | |
| tropomyosin 1 (alpha) | TPM1 |
| TPX2, microtubule- | TPX2 |
| associated protein homolog | |
| (Xenopus laevis) | |
| TTK protein kinase | TTK |
| tubulin, beta polypeptide /// | TUBB /// |
| tubulin, beta polypeptide | MGC8685 |
| paralog | |
| tubulin, gamma 1 | TUBG1 |
| thymidylate synthetase | TYMS |
| ubiquitin-conjugating | UBE2C |
| enzyme E2C | |
| ubiquitin-conjugating | UBE2S |
| enzyme E2S | |
| ubiquitin-like, containing | UHRF1 |
| PHD and RING finger | |
| domains, 1 | |
| unc-5 homolog B (C. elegans) | UNC5B |
| unc-5 homolog B (C. elegans) | UNC5B |
| WD repeat domain 1 | WDR1 |
| WNT1 inducible signaling | WISP1 |
| pathway protein 1 | |
| WNT1 inducible signaling | WISP1 |
| pathway protein 1 | |
| Xg blood group | XG |
| (pseudoautosomal | |
| boundary-divided on the X | |
| chromosome) | |
| zinc finger and BTB domain | ZBTB1 |
| containing 1 | |
| zinc finger protein 36, C3H | ZFP36 |
| type, homolog (mouse) | |
| zinc finger protein 367 | ZNF367 |
| ZW10 interactor | ZWINT |

In some embodiments, the endogenous gene, the expression of which is capable of being modulated by exposure to a BMP is an early gene. In some embodiments, endogenous early genes of this invention may include genes that express during the early stages in a signal process pathway upon exposure to a BMP.

In some embodiments, the early gene is a gene whose expression is modulated within 48 hours after exposure to a BMP. In some embodiments, the early gene is a gene whose expression is modulated within 24 hours after exposure to a BMP. In some embodiments, the early gene is a gene whose expression is modulated within 18 hours after exposure to a BMP. In some embodiments, the early gene is a gene whose expression is modulated within 12 hours after exposure to a BMP. In yet other embodiments, the early gene is a gene whose expression is modulation within 6 hours after exposure to a BMP. In other embodiments, the early gene is a gene whose expression is modulation within 4 hours after exposure to a BMP. In other embodiments, the early gene is a gene whose expression is modulation within 2 hours after exposure to a BMP. In other embodiments, the early gene is a gene whose expression is modulation within 1 hour after exposure to a BMP. In other embodiments, the early gene is a gene whose expression is modulation within 30 minutes after exposure to a BMP.

In some embodiments, the endogenous early gene includes, but is not limited to, Id-1, Id-2, Id-3, Id-4, Msx2, Dlx2, Dlx3, Dlx5, Noggin, Smad6, Smad7 and Runx2. In some embodiments, the endogenous early gene is Id-1 gene. The transcription factor ID-1 is a dominant negative inhibitor of basic helix-loop-helix proteins and is a direct target of BMP. BMP strongly activates the ID-1 promoter and ectopic expression of ID proteins can mimic BMP-induced responses. Accordingly, activation of the Id-1 gene may be useful as an early marker for BMP signal transduction.

In some embodiments, the endogenous gene, the expression of which is capable of being modulated by exposure to a BMP is a late gene. In some embodiments, a late gene is a gene whose expression is modulated more than 48 hours after exposure to a BMP. In some embodiments, a late gene is a gene whose expression is modulated more than 48 hours but within 72 hours after exposure to a BMP. In some embodiments, a late gene is a gene whose expression is modulated more than 48 hours but within 96 hours after exposure to a BMP. In some embodiments, a late gene is a gene whose expression is modulated more than 48 hours but within 120 hours after exposure to a BMP. In some embodiments, a late gene is a gene whose expression is modulated more than 48 hours but within 144 hours after exposure to a BMP. In some embodiments, a late gene is a gene whose expression is modulated more than 48 hours but within 168 hours after exposure to a BMP.

In some embodiments, the late gene includes but is not limited to HEY1, DIO2, ADAMTS9, HAS3, FGFR3, MFI2, CHI3L1, NOG, BAMBI, GREM1, GREM2 and SOST. In some embodiments, the late gene includes but is not limited to FGFR3, DIO2, HEY1, HAS3, ADAMTS9 and MFI2. In other embodiments, the late gene includes but is not limited to NOG, BAMBI, GREM1, and GREM2.

In some embodiments, the expression of the gene is up-regulated by exposure to a BMP. In other embodiments, the expression of the gene is down-regulated by exposure to a BMP.

In some embodiments, quantitative real-time polymerase chain reaction (QPCR) is used to amplify the cDNA of the endogenous gene in sample-treated or untreated cells. QPCR is a very sensitive method that allows detection of infinitely small changes in the transcription of a gene of interest. Accordingly, QPCR-based assays are highly sensitive and may be useful for detecting small amounts of neutralizing antibodies present in biological samples. Methods of quantitative real-time PCR using TaqMan probes are well known in the art. Detailed protocols for real-time quantitative PCR are described in, for example, Gibson et al., “A novel method for real time quantitative RT-PCR”, Genome Res., 10:995-1001 (1996) and Heid et al., “Real time quantitative PCR”, Genome Res., 10:986-994 (1996).

In some embodiments, the samples of this invention may be prescreened to determine if antibodies specific for the BMP of interest are present prior to determining whether the antibodies are neutralizing. The samples of this invention may also have tested positive in a primary neutralizing antibody assay and are now being subjected to the assay of this invention as a confirmatory assay for the presence of neutralizing antibodies. In some embodiments, the samples are prescreened with an immunoassay, such as an ELISA assay. In other embodiments, the samples are prescreened with a cell-based assay, such as, for example, the activation of a reporter gene. The reporter gene may be the luciferase gene. The luciferase gene may be linked to a promoter of a gene that is capable of being modulated by exposure to a BMP. Such a gene may be an early or late gene, including, but not limited to, Id-1, Id-2, Id-3, Id-4, Msx2, Dlx2, Dlx3, Dlx5, Noggin, Smad6, Smad7, Runx2, fibromodulin, Hey 1 and SFRP-2. In some embodiments, the samples are prescreened with a cell-based assay, such as, for example, an assay that involves monitoring alkaline phosphatase activity, for example, in rat osteosarcoma (ROS) cells.

In some embodiments of this invention, the assays are conducted on 96-well plates containing the test samples and the control samples on the same assay plate. In other embodiments, the assays are conducted by high-throughput screening.

Practice of the invention will be still more fully understood from the following examples, which are presented herein for illustration only and should not be construed as limiting the invention in any way.

### Example 1

### Detection of Neutralizing Antibodies to OP-1 in Human Serum Samples by Quantitative PCR (QPCR)

A549 cells (ATCC Cat. #CCL-185) in medium containing 1% FBS (F-12K medium (ATCC Cat. #30-2004) containing 1% FBS) were plated on 96-well tissue culture microtiter plates at an optimal seeding density and incubated at 37° C. until the cells were stably attached to the plate in a monolayer. This may take a few hours and up to 24 hours depending on the cell type. In the case of A549 cells, they attach to the plate by 24 hours.

Positive controls consisting of a pre-determined effective BMP concentration spiked in normal human serum (NHS) pool were prepared by mixing 97.5 μL NHS pool with 2.5 μL of the 24 μg/mL OP-1 spike.

Positive controls consisting of an anti-BMP antibody (12G3 monoclonal antibody) at 40 μg/mL (“12G3/1000” in NHS was prepared as follows. The 12G3 monoclonal antibody was diluted to 200 μg/mL in pooled NHS. The antibody was then further diluted to 40 μg/mL in NHS in a total volume of 97.5 μL. A 2.5 μL volume of the 24 μg/mL OP-1 spike was then added to the mixture.

Positive controls consisting of an anti-BMP antibody (12G3 monoclonal antibody) at 20 μg/mL “12G3/500” in NHS was prepared as follows. The 12G3 monoclonal antibody was diluted to 200 μg/mL in pooled NHS. The antibody was then further diluted to 20 μg/mL in NHS in a total volume of 97.5 μL. A 2.5 μL volume of the 24 μg/mL OP-1 spike was then added to the mixture.

Samples were spiked with the “effective BMP concentration” and allowed to pre-incubate for a minimum of 30 minutes prior to cell stimulation.

The “effective BMP concentration” was determined by incubating either 15 ng/mL or 25 ng/mL of BMP-7 (each done in triplicate) with increasing concentrations of an anti-BMP-7 monoclonal antibody (12G3). The relative quantity (RQ) values (discussed in more detail below) for the two concentrations were compared and BMP-7 at the concentration of 15 ng/mL was determined to be the effective concentration.

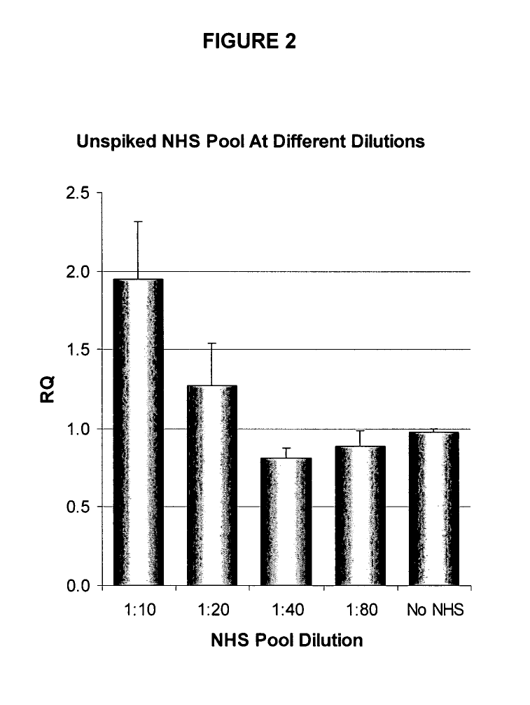

The controls and samples were all diluted to the minimal required dilution (MRD) and added in a minimum of duplicates to the plates containing the pre-plated cells. The MRD was determined by testing unspiked NHS pool at different dilutions (1:10, 1:20, 1:40, 1:80) and determining the minimum dilution at which the serum samples display background RQ values similar to control samples containing no serum (No NHS). As illustrated in FIG. 2, the MRD was determined to be 1:40.

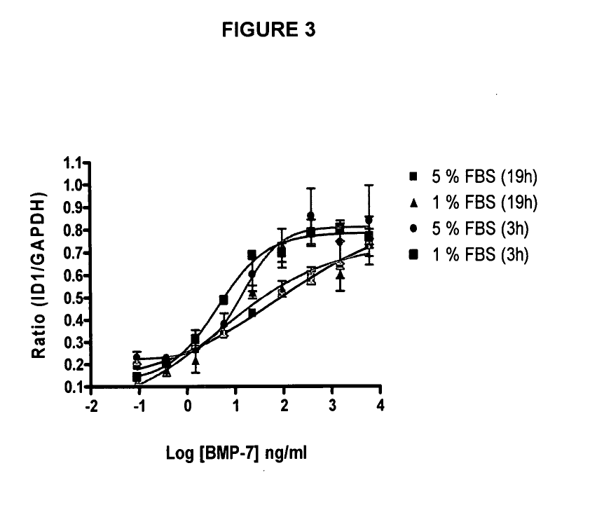

Following sample addition, plates were incubated at 37° C. for 3 hours. The 3 hour time period of incubation was an optimized time period chosen based on a study comparing different incubation time periods on the ratio of target/housekeeping (Id-1/GAPDH) gene expression. Briefly, cells were incubated for either 19 hours or 3 hours with increasing concentrations of BMP-7 and the effect on the ratio of target/housekeeping (Id-1/GAPDH) gene expression was determined. The 3 hour time period of incubation was chosen as the optimized time period based on the response curve, as illustrated in FIG. 3. The concentration of FBS to used in the assay was also optimized based on a study comparing different concentrations of FBS on the ratio target/housekeeping (Id-1/GAPDH) gene expression. Briefly, cells were incubated for 3 hours with increasing concentrations of BMP-7 in either 1% or 5% FBS and the effect on the ratio of target/housekeeping (Id-1/GAPDH) gene expression was determined. The 1% FBS was chosen as the optimized condition based on the response curve, as illustrated in FIG. 3.

Cells were lysed and Poly A+ mRNA was isolated using 96 well Turbo Capture plates (Qiagen, Valencia, Calif.).

Isolated mRNA was then used as template for cDNA synthesis in a reverse transcription reaction. Samples were first adjusted to 25° C. for 5 minutes, then reverse transcription was carried out at 42° C. for 30 minutes. Samples were then incubated at 85° C. for 5 minutes to denature the RT enzyme, and then cooled to 4° C.

Target (Id-1) and housekeeping (GAPDH) gene expression were measured by QPCR using pre-validated and gene-specific TaqMan reagents (Applied Biosystems, Foster City, Calif.). Each cDNA sample was analyzed in triplicate wells for each of the two genes for a total of six QPCR reactions per cDNA sample and 12 reactions per serum sample. PCR reactions could be carried out for example in an ABI 7900HT Fast Real-Time PCR system using TaqMan Universal PCR Master Mix. Target (Id-1) and housekeeping (GAPDH) gene expression was quantified using the delta delta Ct method (Livak and Schmittgen, 2001). In brief, the PCR cycle at which amplification of the gene crosses the threshold (Ct) was recorded for both the target (Id-1) and housekeeping (GAPDH) genes. The average delta Ct of each cell culture well was calculated by subtracting the average Ct of the three housekeeping gene replicates from the average Ct of the three target gene replicates. The delta Ct of a calibrator sample, consisting of BMP spiked into the NHS pool, was then subtracted from the delta Ct of each unknown, yielding the delta delta Ct. The delta delta Ct of each unknown is expressed relative to the delta delta Ct of the calibrator sample, and was referred to as the Relative Quantity or RQ value. The mean, standard deviation and percent CV of the RQ values from the treatment replicates were then calculated and reported.

As shown in Table 2, the target Id-1 gene and the housekeeping GAPDH gene were amplified with similar efficiencies. This is an important feature because similar efficiencies of the target and housekeeping genes is necessary for normalization of gene expression.

TABLE 2

| QPCR | ID-1 | | GAPDH | |
|---|---|---|---|---|
| Run | slope | Efficiency | slope | Efficiency |
| 1 | −3.34 | 99% | −3.26 | 103% |
| 2 | −3.49 | 94% | −3.06 | 112% |
| 3 | −3.45 | 95% | −3.38 | 97% |
| 4 | −3.70 | 86% | −3.37 | 98% |
| 5 | −3.34 | 99% | −3.41 | 96% |
| 6 | −3.15 | 108% | −3.34 | 99% |
| 7 | −3.61 | 89% | −3.51 | 93% |
| Average | | 96% | | 100% |

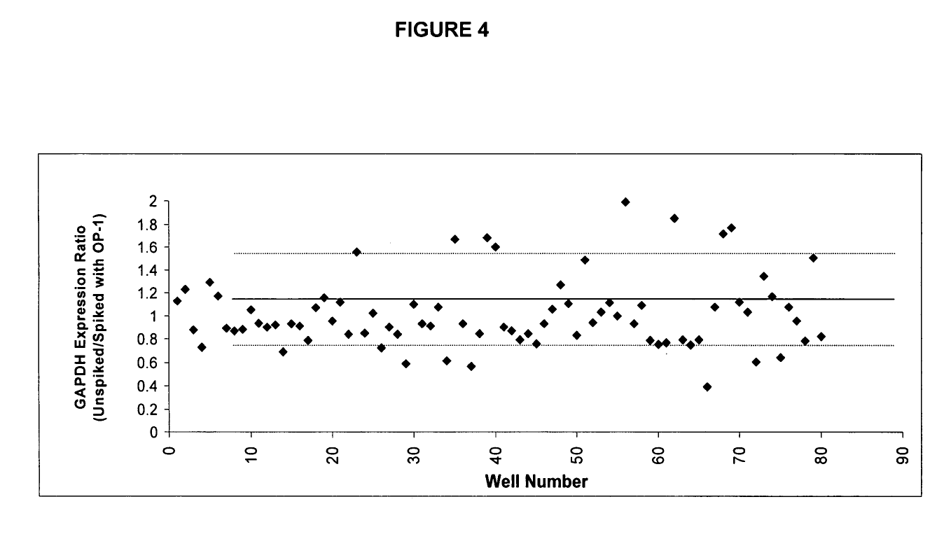

OP-1 (BMP-7) also did not affect the expression of the housekeeping GAPDH gene. As set forth in FIG. 4, the GAPDH expression ratio (unspiked/spiked with OP-1) was relatively uniform in 80 different samples tested.

### Example 2

### Cut Point Determination

The assay cut point was determined using data from an experiment conducted during the assay validation. Data from this study are summarized in Table 3, below.

TABLE 3
Cut Point Determination Data from Experiment

| | PLATE 1 | | PLATE 2 | | PLATE 3 | |
|---|---|---|---|---|---|---|
| | Avg RQ | SD RQ | Avg RQ | SD RQ | Avg RQ | SD RQ |
| Unspiked | 0.22 | 0.00 | 0.27 | 0.02 | 0.28 | 0.03 |
| Avg of Spike | 1.00 | | 1.00 | | 1.00 | |
| Controls | | | | | | |
| 12G3 1000 | 0.51 | 0.03 | 0.49 | 0.02 | 0.54 | 0.06 |
| 12G3 500 | 0.63 | 0.08 | 0.86 | 0.08 | 0.74 | 0.04 |
| S1 | 1.18 | | 1.12 | | 1.95 | |
| S2 | 1.33 | | 1.15 | | 1.15 | |
| S3 | 1.44 | | 1.40 | | 1.33 | |
| S4 | 1.26 | | 1.69 | | 1.26 | |
| S5 | 1.26 | | 1.59 | | 1.50 | |
| S6 | 1.15 | | 1.33 | | 1.52 | |
| S7 | 1.42 | | 1.33 | | 1.31 | |
| S8 | 1.23 | | 1.51 | | 1.32 | |
| S9 | 1.17 | | 1.55 | | 1.32 | |
| S10 | 1.40 | | 1.63 | | 1.44 | |
| S11 | 0.87 | | 1.15 | | 1.25 | |
| S12 | 1.12 | | 1.49 | | 1.03 | |
| S13 | 1.23 | | 1.57 | | 1.47 | |
| S14 | 1.08 | | 1.57 | | 1.12 | |
| S15 | 1.08 | | 1.44 | | 1.22 | |
| S16 | 1.23 | | 1.46 | | 1.28 | |
| S17 | 1.27 | | 1.54 | | 1.16 | |
| S18 | 1.29 | | 1.43 | | 0.98 | |
| S19 | 1.13 | | 1.30 | | 1.08 | |
| S20 | 1.49 | | 1.62 | | 1.34 | |
| S21 | 0.94 | | 1.26 | | 1.35 | |
| S22 | 1.18 | | 1.80 | | 1.04 | |
| S23 | 1.26 | | 1.50 | | 1.62 | |
| S24 | 1.13 | | 1.78 | | 1.17 | |
| S25 | 1.25 | | 1.71 | | 1.11 | |
| S26 | 1.28 | | 1.26 | | 1.26 | |
| S27 | 1.33 | | 1.40 | | 1.00 | |
| S28 | 1.24 | | 1.24 | | 1.26 | |
| S29 | 1.20 | | 1.48 | | 1.16 | |
| S30 | 1.20 | | 1.69 | | 1.09 | |
| S31 | 1.06 | | 1.25 | | 1.28 | |
| S32 | 1.33 | | 1.33 | | 1.18 | |
| S33 | 1.32 | | 1.32 | | 1.36 | |
| S34 | 1.22 | | 1.19 | | 1.07 | |
| S35 | 1.35 | | 1.36 | | 1.18 | |
| S36 | 1.42 | | 1.03 | | 1.28 | |
| S37 | 1.29 | | 1.29 | | 1.23 | |
| S38 | 1.08 | | 1.63 | | 1.35 | |
| S39 | 1.29 | | 1.54 | | 1.14 | |
| S40 | 1.25 | | 1.54 | | 1.20 | |
| S41 | 0.93 | | 1.25 | | 1.41 | |
| S42 | 1.07 | | 0.96 | | 1.44 | |
| S43 | 0.97 | | 1.15 | | 1.31 | |
| S44 | 1.19 | | 1.33 | | 1.11 | |
| S45 | 1.16 | | 1.54 | | 1.66 | |
| S46 | 0.99 | | 1.37 | | 1.10 | |
| S47 | 1.09 | | 1.70 | | 1.40 | |
| S48 | 1.08 | | 2.14 | | 1.65 | |
| S49 | 1.05 | | 1.64 | | 1.36 | |
| S50 | 0.75 | | 1.58 | | 1.53 | |
| Mean RQ | 1.19 | | 1.44 | | 1.29 | |
| StDev RQ | 0.151 | | 0.222 | | 0.192 | |
| 95% LCL | 0.942 | | 1.077 | | 0.971 | |

| | |
|---|---|
| Mean 95% | 0.997 |
| LCL | |

The assay cut point was determined by testing fifty (50) different NHS samples, spiked with 15 ng/ml of OP-1. Each sample was tested in singlicate so that all fifty samples could be evaluated on the same tissue culture plate. Three such plates (Plate 1, Plate 2 and Plate 3) were set up on the same day so that plate to plate variation could be taken into account when determining the assay cut point. The RQ value for each sample was calculated using the delta delta Ct method, with a control solution consisting of 15 ng/mL OP-1 in pooled NHS as a reference. The mean RQ value from all four replicates of this control solution on each plate was set as the reference.

Three statistical tests (KS, D'Agostino & Pearson, and Shapiro & Wilk) were run in order to assess the normality of the RQ distribution for each plate, shown in Table 4 below. In the case of Plate 1 and Plate 2, all three tests showed that the RQ distribution is normal. For Plate 3, only one out of the three tests showed that the RQ distribution is normal. Taken together however, these results trend towards normality. As a result, the cut point for this assay was determined using a parametric method.

TABLE 4

| | PLATE 1 | PLATE 2 | PLATE 3 |
|---|---|---|---|
| Number of values | 50 | 50 | 50 |
| Mean | 1.191 | 1.443 | 1.287 |
| Std. Deviation | 0.151 | 0.2212 | 0.1917 |
| Std. Error | 0.02136 | 0.03129 | 0.02711 |
| KS normality test | | | |
| KS distance | 0.08291 | 0.0746 | 0.1109 |
| P value | P > 0.10 | P > 0.10 | P > 0.10 |
| Passed normality test (alpha = 0.05)? | Yes | Yes | Yes |
| D'Agostino & Pearson | | | |
| omnibus normality test | | | |
| K2 | 3.634 | 2.742 | 11.95 |
| P value | 0.1625 | 0.2539 | 0.0025 |
| Passed normality test (alpha = 0.05)? | Yes | Yes | No |
| Shapiro-Wilk normality test | | | |
| W | 0.9772 | 0.9797 | 0.9445 |
| P value | 0.4394 | 0.5395 | 0.0203 |
| Passed normality test (alpha = 0.05)? | Yes | Yes | No |

The lower 95% confidence limit (95% LCL) for the RQs from each plate was determined. The mean 95% LCL for the three plates in this study was then calculated. This value was found to be 0.997 and designated as the cut point for this assay. Samples with a RQ lower than or equal to 0.997 were considered positive for the presence of anti-OP-1 neutralizing antibodies. Samples with a RQ value higher than 0.997 were considered negative.

### Example 3

### Validation of Intermediate Precision

Intermediate precision was examined by testing six (6) individually prepared 15 ng/ml OP-1 spikes on a minimum of one (1) plate over three (3) days. Each of the six samples was tested in duplicate wells in the tissue culture plate. RQ values were calculated using the delta delta Ct method. The mean RQ value from four replicates of a control solution consisting of 15 ng/mL OP-1 in pooled NHS was set as the reference. The mean RQ as well as the percent difference from the mean for each duplicate wells was also calculated.

Data from the intermediate precision study are summarized in Table 5, below.

TABLE 5
Intermediate Precision (Inter-Assay Precision)

| | Plate | | | QPCR Plate | Mean | % Difference | | Plate | Plate | Plate |
|---|---|---|---|---|---|---|---|---|---|---|
| | ID | | Sample ID | Sample ID | RQ | from the Mean | Status | Mean RQ | RQ StDev | RQ % CV |
| Experiment 1 | | Controls | Unspiked | 142 | 0.290 | 5.173 | Valid | | | |
| | | | Control Reference | 144 | 1.000 | NA | Valid | | | |
| | | | 1000 ng/ml 12G3 | 148 | 0.497 | 6.505 | Valid | | | |
| | | | 500 ng/ml 12G3 | 150 | 0.664 | 0.355 | Valid | | | |
| | | Samples | Spike 1 | 176 | 1.193 | 4.925 | Valid | 1.219 | 0.061 | 5.025 |
| | | | Spike 2 | 178 | 1.323 | 7.707 | Valid | | | |
| | | | Spike 3 | 180 | 1.182 | 4.421 | Valid | | | |
| | | | Spike 4 | 182 | 1.145 | 8.286 | Valid | | | |
| | | | Spike 5 | 184 | 1.234 | 2.732 | Valid | | | |
| | | | Spike 6 | 186 | 1.235 | 12.071 | Valid | | | |
| Experiment 2 | | Controls | Unspiked | 256 | 0.227 | 4.034 | Valid | | | |
| | | | Control Reference | 258 | 1.000 | NA | Valid | | | |
| | | | 1000 ng/ml 12G3 | 262 | 0.494 | 0.455 | Valid | | | |
| | | | 500 ng/ml 12G3 | 264 | 0.665 | 7.168 | Valid | | | |
| | | Samples | Spike 1 | 266 | 0.982 | 9.638 | Valid | 1.018 | 0.125 | 12.251 |
| | | | Spike 2 | 268 | 1.175 | 3.345 | Valid | | | |
| | | | Spike 3 | 270 | 0.879 | 3.255 | Valid | | | |
| | | | Spike 4 | 272 | 1.084 | 1.908 | Valid | | | |
| | | | Spike 5 | 274 | 1.110 | 0.184 | Valid | | | |
| | | | Spike 6 | 276 | 0.878 | 6.948 | Valid | | | |
| Experiment 3 | | Controls | Unspiked | 380 | 0.285 | 18.539 | Valid | | | |
| | | | Control Reference | 382 | 1.000 | NA | Valid | | | |
| | | | 1000 ng/ml 12G3 | 386 | 0.490 | 3.019 | Valid | | | |
| | | | 500 ng/ml 12G3 | 388 | 0.726 | 4.190 | Valid | | | |
| | | Samples | Spike 1 | 426 | 1.158 | 1.317 | Valid | 1.159 | 0.055 | 4.769 |
| | | | Spike 2 | 428 | 1.083 | 7.178 | Valid | | | |
| | | | Spike 3 | 430 | 1.103 | 1.504 | Valid | | | |
| | | | Spike 4 | 432 | 1.212 | 0.173 | Valid | | | |
| | | | Spike 5 | 434 | 1.193 | 4.085 | Valid | | | |
| | | | Spike 6 | 436 | 1.208 | 3.835 | Valid | | | |

| Intermediate Precision Analysis | |
|---|---|
| Mean RQ across 3 plates | 1.132 |
| StDev RQ across 3 plates | 0.119 |
| % CV RQ acroos 3 plates | 10.484 |
| Acceptance criteria status | Pass |

All 15 ng/ml OP-1 spikes analyzed in the Intermediate Precision study were valid based on the criteria that the percent difference from the mean of duplicate treatment wells≦30%.

The Mean RQ of all valid spikes, across three days, is equal to 1.132. The standard deviation and CV of the valid spikes are equal to 0.119 and 10.484, respectively. Intermediate Precision for this assay was deemed acceptable if the RQ for the spikes generate a % CV less than or equal to 30. Based on this pre-defined acceptance criteria, the Intermediate precision of this assay was deemed acceptable.

### Example 4

### Validation of Repeatability

Intra-assay precision was examined by testing six (6) individually prepared 15 ng/ml OP-1 spikes on three (3) plates on one (1) day, tested by one (1) analyst. Each of the six samples was tested in duplicate wells in the tissue culture plate. RQ values were calculated using the delta delta Ct method. The mean RQ value from four replicates of a control solution consisting of 15 ng/mL OP-1 in pooled NHS was set as the reference. The mean RQ as well as the percent difference from the mean for each duplicate wells was also calculated.

Data from the intermediate precision study are summarized in Table 6, below.

TABLE 6
Repeatability (Intra-assay Precision) from Experiment

| | Plate | | | QPCR Plate | Mean | % Difference | | Plate | Plate | Plate |
|---|---|---|---|---|---|---|---|---|---|---|
| | ID | | Sample ID | Sample ID | RQ | from the Mean | Status | Mean RQ | RQ StDev | RQ % CV |
| Plate 1 | | Controls | Unspiked | 256 | 0.227 | 4.034 | Valid | | | |
| | | | Control Reference | 258 | 1.000 | NA | Valid | | | |
| | | | 1000 ng/ml 12G3 | 262 | 0.494 | 0.455 | Valid | | | |
| | | | 500 ng/ml 12G3 | 264 | 0.665 | 7.168 | Valid | | | |
| | | Samples | Spike 1 | 266 | 0.982 | 9.638 | Valid | 1.018 | 0.125 | 12.251 |
| | | | Spike 2 | 268 | 1.175 | 3.345 | Valid | | | |
| | | | Spike 3 | 270 | 0.879 | 3.255 | Valid | | | |
| | | | Spike 4 | 272 | 1.084 | 1.908 | Valid | | | |
| | | | Spike 5 | 274 | 1.110 | 0.184 | Valid | | | |
| | | | Spike 6 | 276 | 0.878 | 6.948 | Valid | | | |
| Plate 2 | | Controls | Unspiked | 278 | 0.201 | 8.672 | Valid | | | |
| | | | Control Reference | 280 | 1.000 | NA | Valid | | | |
| | | | 1000 ng/ml 12G3 | 284 | 0.457 | 5.152 | Valid | | | |
| | | | 500 ng/ml 12G3 | 286 | 0.556 | 6.258 | Valid | | | |
| | | Samples | Spike 1 | 288 | 0.981 | 7.062 | Valid | 0.973 | 0.089 | 9.154 |
| | | | Spike 2 | 290 | 1.068 | 4.575 | Valid | | | |
| | | | Spike 3 | 292 | 0.845 | 2.184 | Valid | | | |
| | | | Spike 4 | 294 | 1.078 | 1.451 | Valid | | | |
| | | | Spike 5 | 296 | 0.934 | 0.960 | Valid | | | |
| | | | Spike 6 | 298 | 0.933 | 0.077 | Valid | | | |
| Plate 3 | | Controls | Unspiked | 300 | 0.245 | 5.523 | Valid | | | |
| | | | Control Reference | 302 | 1.000 | | Valid | | | |
| | | | 1000 ng/ml 12G3 | 306 | 0.564 | 5.318 | Valid | | | |
| | | | 500 ng/ml 12G3 | 308 | 0.679 | 3.194 | Valid | | | |
| | | Samples | Spike 1 | 310 | 0.957 | 9.172 | Valid | 1.061 | 0.113 | 10.610 |
| | | | Spike 2 | 312 | 1.019 | 4.923 | Valid | | | |
| | | | Spike 3 | 314 | 1.180 | 5.258 | Valid | | | |
| | | | Spike 4 | 316 | 1.210 | 9.664 | Valid | | | |
| | | | Spike 5 | 318 | 1.059 | 13.914 | Valid | | | |
| | | | Spike 6 | 320 | 0.941 | 10.054 | Valid | | | |

| Repeatability Analysis | |
|---|---|
| Mean RQ across 3 plates | 1.017 |
| StDev RQ across 3 plates | 0.110 |
| % CV RQ across 3 plates | 10.767 |
| Acceptance criteria status | Pass |

All 15 ng/ml OP-1 spikes analyzed in the intra-assay precision study were valid based on the criteria that the percent difference from the mean of duplicate treatment wells≦30%.

The Mean RQ of all valid spikes, across the three plates, is equal to 1.017. The standard deviation and % CV of the valid spikes are equal to 0.110 and 10.767, respectively. Intra-assay precision was considered acceptable for this assay if the Mean RQ of each valid spike was within ±30% of the Mean RQ for all valid spikes on each individual plate. Based on this pre-defined acceptance criteria, the intra-assay precision was deemed acceptable.

### Example 5

### Validation of Sensitivity

The limit of detection parameter validated the lowest anti-OP-1 antibody concentration that yields a measurable neutralizing affect in this assay. Limit of detection (sensitivity) was examined by preincubating OP-1 spikes (15 ng/ml) with four (4) different 12G3 antibody concentrations (75, 150, 250 and 500 ng/ml). Each 12G3 concentration was evaluated in six (6) NHS samples per plate over three plates in one day. As for other parameters discussed above, these samples were tested in duplicate wells. RQ values were calculated using the delta delta Ct method. The mean RQ value from four replicates of a control solution consisting of 15 ng/mL OP-1 in pooled NHS was set as the reference. The mean RQ as well as the percent difference from the mean for each duplicate wells was also calculated.

Data from the limit of detection (sensitivity) study are summarized in Table 7, below.

TABLE 7
Sensitivity Data from Experiment

| | | Plate ID | | | | | | | | | | | | |
|---|---|---|---|---|---|---|---|---|---|---|---|---|---|---|
| | | PLATE 1 % | | | | PLATE 2 % | | | | PLATE 3 % | | | | |
| | | QPCR | | Difference | | QPCR | | Difference | | QPCR | | Difference | | Sensitivity |
| | | Sample | Mean | from the | | Sample | Mean | from the | | Sample | Mean | from the | | Acceptance |
| | Sample ID | Number | RQ | Mean | Status | Number | RQ | Mean | Status | Number | RQ | Mean | Status | Criteria |
| Unspiked | | 438 | 0.271 | 9.459 | Valid | 486 | 0.309 | 2.950 | Valid | 546 | 0.244 | 0.926 | Valid | NA |
| Control Reference | | 440 | 1.000 | | Valid | 488 | 1.000 | | Valid | 548 | 1.000 | | Valid | |
| 1000 ng/ml 12G3 | | 446 | 0.613 | 16.667 | Valid | 494 | 0.680 | 8.853 | Valid | 554 | 0.605 | 11.144 | Valid | |
| 500 ng/ml 12G3 | | 448 | 0.800 | 6.206 | Valid | 496 | 0.822 | 5.124 | Valid | 556 | 0.790 | 15.898 | Valid | |
| 75 | Replicate 1 | 450 | 0.767 | 2.841 | Valid | 498 | 0.808 | 10.029 | Valid | 558 | 0.826 | 0.430 | Valid | FAIL |
| | Replicate 2 | 452 | 0.742 | 15.246 | Valid | 500 | 0.812 | 5.718 | Valid | 560 | 0.826 | 3.636 | Valid | |
| | Replicate 3 | 454 | 0.751 | 17.627 | Valid | 502 | 0.840 | 12.474 | Valid | 562 | 0.902 | 10.131 | Valid | |
| | Replicate 4 | 456 | 0.694 | 1.943 | Valid | 504 | 0.932 | 5.292 | Valid | 564 | 0.903 | 6.692 | Valid | |
| | Replicate 5 | 458 | 0.823 | 11.390 | Valid | 506 | 0.877 | 1.911 | Valid | 566 | 0.940 | 2.200 | Valid | |
| | Replicate 6 | 460 | 1.022 | 10.630 | Valid | 508 | 0.854 | 2.845 | Valid | 568 | 0.970 | 7.605 | Valid | |
| 150 | Replicate 1 | 462 | 0.916 | 6.848 | Valid | 510 | 0.803 | 5.588 | Valid | 570 | 0.985 | 9.419 | Valid | FAIL |
| | Replicate 2 | 464 | 0.787 | 4.820 | Valid | 512 | 0.758 | 6.698 | Valid | 572 | 0.891 | 2.750 | Valid | |
| | Replicate 3 | 466 | 0.825 | 4.785 | Valid | 514 | 0.666 | 4.583 | Valid | 574 | 0.980 | 14.281 | Valid | |
| | Replicate 4 | 468 | 0.669 | 11.992 | Valid | 516 | 0.693 | 0.900 | Valid | 576 | 1.014 | 7.046 | Valid | |
| | Replicate 5 | 470 | 0.900 | 5.398 | Valid | 518 | 0.721 | 9.878 | Valid | 578 | 1.008 | 5.139 | Valid | |
| | Replicate 6 | 472 | 0.901 | 3.833 | Valid | 520 | 0.657 | 6.706 | Valid | 580 | 0.971 | 7.931 | Valid | |
| 250 | Replicate 1 | 486 | 0.968 | 3.914 | Valid | 522 | 1.008 | 7.827 | Valid | 582 | 1.036 | 10.333 | Valid | FAIL |
| | Replicate 2 | 488 | 0.995 | 5.348 | Valid | 524 | 0.846 | 10.075 | Valid | 584 | 0.876 | 15.311 | Valid | |
| | Replicate 3 | 490 | 0.934 | 1.466 | Valid | 526 | 0.854 | 1.383 | Valid | 586 | 0.769 | 0.219 | Valid | |
| | Replicate 4 | 492 | 1.003 | 8.746 | Valid | 528 | 0.965 | 0.348 | Valid | 588 | 0.803 | 0.772 | Valid | |
| | Replicate 5 | 494 | 1.075 | 4.398 | Valid | 530 | 0.912 | 3.245 | Valid | 590 | 0.812 | 2.348 | Valid | |
| | Replicate 6 | 496 | 0.788 | 0.706 | Valid | 532 | 0.596 | 1.143 | Valid | 592 | 0.885 | 8.909 | Valid | |
| 500 | Replicate 1 | 474 | 0.770 | 2.253 | Valid | 534 | 0.610 | 4.047 | Valid | 594 | 0.674 | 7.467 | Valid | PASS |
| | Replicate 2 | 476 | 0.811 | 6.633 | Valid | 536 | 0.667 | 20.873 | Valid | 596 | 0.775 | 8.101 | Valid | |
| | Replicate 3 | 478 | 0.817 | 1.641 | Valid | 538 | 0.713 | 12.537 | Valid | 598 | 0.767 | 6.231 | Valid | |
| | Replicate 4 | 480 | 0.791 | 3.039 | Valid | 540 | 0.492 | 0.740 | Valid | 600 | 0.743 | 8.715 | Valid | |
| | Replicate 5 | 482 | 0.783 | 2.896 | Valid | 542 | 0.613 | 1.264 | Valid | 602 | 0.772 | 4.689 | Valid | |
| | Replicate 6 | 484 | 0.843 | 14.039 | Valid | 544 | 0.626 | 5.017 | Valid | 604 | 0.751 | 2.923 | Valid | |

The results for limit of detection (sensitivity) meet the acceptance criteria for plate validity.

Four concentrations of 12G3 were evaluated in this study. These were 75 ng/ml, 150 ng/ml, 250 ng/ml and 500 ng/ml. All of 12G3 spikes analyzed in the limit of detection (sensitivity) study were valid based on the criteria that the percent difference from the mean of duplicate treatment wells≦30%. Limit of detection (sensitivity) is acceptable if all valid spikes have a Mean RQ value below the plate specific cut point. The cut point for this assay was determined to be an RQ value of 0.997. As shown in Table 7, the Mean RQ of all the 500 ng/ml 12G3 spikes, across three plates, on one day, is <0.997. The other three 12G3 concentrations (75, 150 and 250 ng/ml) did not meet the acceptance criteria for this parameter (specific replicates with a mean RQ higher than the cut point (i.e., 0.997) are underlined in Table 7).

Based on the pre-defined acceptance criteria, the acceptable limit of detection (sensitivity) of the assay is 500 ng/ml.

### Example 6

### Gene Expression Analysis by Quantitative RT-PCR

Primary human bone-marrow derived mesenchymal stem cells (hMSC) and hMSC culture media, including Mesenchymal Stem Cell Growth Medium (MSCGM) and Osteogenic Differentiation Medium (ODM), were purchased from Lonza (Walkersville, Md.). Cells were expanded in vitro and used for experimentation within five passages of the initial thaw. BMP-7 treatments were applied in ODM. ODM was prepared according to the manufacturer's instructions using the provided supplements of ascorbic acid and beta glycerophosphate but excluding the dexamethasone. The concentration of FBS in MSCGM and ODM was approximately 10%. BMP-7 was diluted in ODM to the indicated concentrations.

Primary hMSC were seeded at 1.5×10⁴ cells per cm² in T-75 tissue culture flasks. Twenty four hours later, cells were treated with ODM alone or ODM containing either 40 ng/ml or 400 ng/ml of BMP-7. Cells were harvested after various time points (1, 2, 3, 4, 5, 6 and 7 days) post BMP-7 treatment and processed for RT-QPCR analysis.

RNA from control (i.e., ODM alone) and BMP-7 treated human mesenchymal stem cells (hMSC) was isolated using the TurboCapture 96 mRNA Kit (Qiagen, Valencia, Calif.) according to the manufacturer's protocol. Reverse transcription was performed using 40 units of M-MLV Reverse Transcriptase (Promega, Madison, Wis.) in a buffer containing 20 mM Tris-HCl, 50 mM KCl, 5 mM MgCl₂, 500 μM each dNTP (Invitrogen, Carlsbad, Calif.) and 5 ng/μl Random Primers (Promega, Madison, Wis.). Reverse transcription was carried out at 23° C. for 10 minutes, 42° C. for 50 minutes followed by a 5 minute inactivation step at 85° C. All reagents and instrumentation for gene expression analysis were obtained from Applied Biosystems (ABI, Foster City, Calif.). Quantitative PCR was carried out using a 7900HT Fast Real-Time PCR System and pre-designed TaqMan Gene Expression Assays according to the manufacturer's specifications. Expression of GAPDH, HEY1, DIO2, ADAMTS9, HAS3, FGFR3, MFI2, CHI3L1, NOG (Noggin), BAMBI, GREM1, GREM2 and SOST gene expression was measured using the standard curve method of relative quantification, according to Applied Biosystems' recommended procedure.

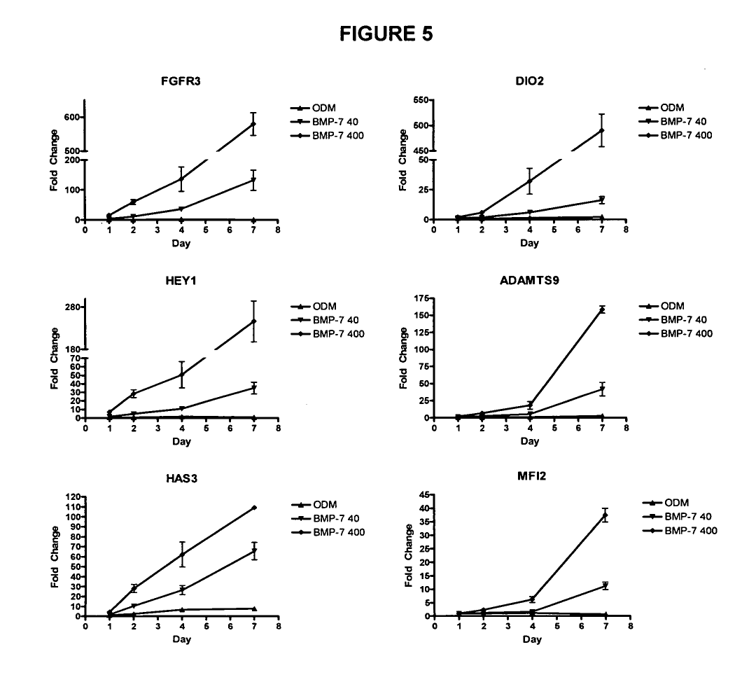

BMP-7 (OP-1) strongly upregulated FGFR3, DIO2, HEY1, HAS3, ADAMTS9 and MFI2 genes in hMSC. The modulation of expression of these genes following BMP-7 treatment was confirmed by QPCR. All six genes showed a dose-responsive increase in expression in BMP-7 treated hMSC over seven days, with a maximum upregulation of approximately 580-fold (FGFR3), 490-fold (DIO2), 250-fold (HEY1), 160-fold (ADAMTS9), 110-fold (HAS3) and 40-fold (MFI2). The extreme magnitude of upregulation observed for these genes suggest that they may play an important role in BMP-7 mediated osteoblastic differentiation. See FIG. 5.

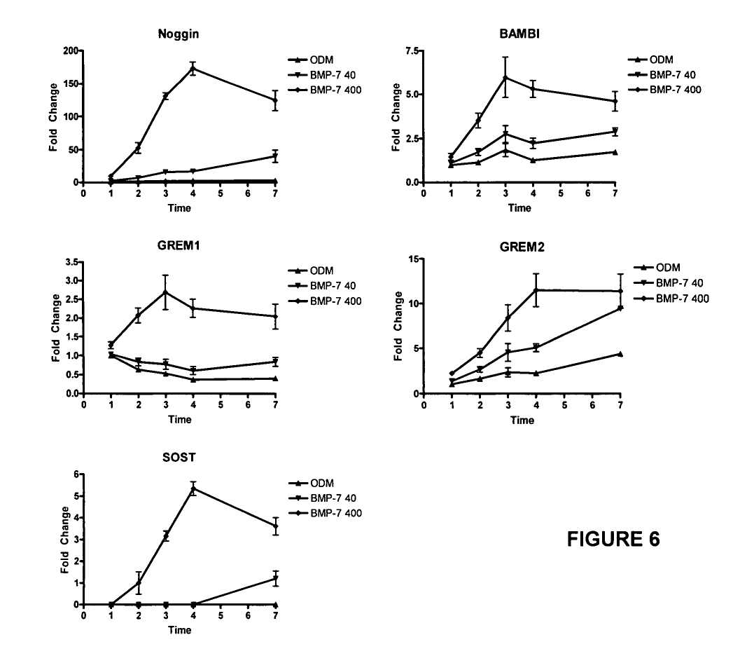

BMP-7 also significantly upregulated the expression of the BMP inhibitors Noggin, BAMBI, GREM1 and GREM2 genes in human mesenchymal stem cells. See FIG. 6.

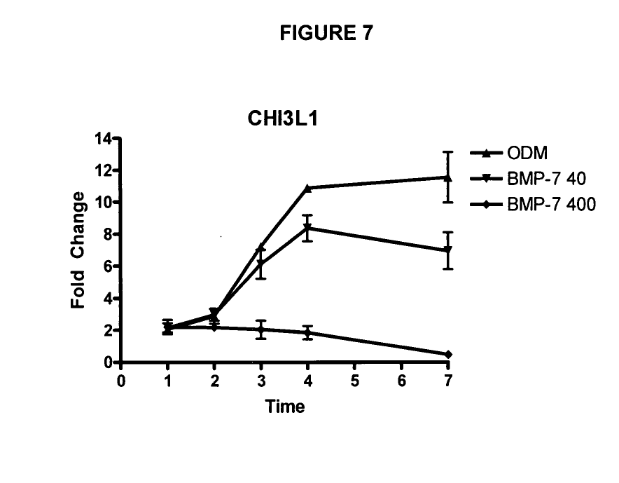

BMP-7 downregulated CHI3L1 (cartilage glycoprotein-39/YKL-40/chitinase 3-like 1) gene expression in human mesenchymal stem cells. The modulation of expression of this gene by BMP-7 treatment was confirmed by QPCR. CHI3L1 was continuously downregulated in a dose-dependent manner in hMSC treated with BMP-7 for seven days, with a maximum 24-fold downregulation observed at the higher dose of BMP-7 relative to untreated cells. See FIG. 7.

What is claimed is:

1. A method for the detection of neutralizing antibodies to a bone morphogenetic protein (BMP) in a sample comprising the steps of:
(a) contacting said sample with a BMP;
(b) incubating said sample from step (a) with a BMP-responsive cell comprising at least one endogenous gene, the expression of which is capable of being modulated by exposure to said BMP;
(c) isolating mRNA from said cell;
(d) preparing cDNA corresponding to said mRNA by reverse transcription;
(e) amplifying said gene from said cDNA of step (d) by quantitative real-time polymerase chain reaction (QPCR);
(f) determining the amount of said gene amplified in step (e) in said cells;
(g) determining the amount of said gene amplified according to step (e) in control cells contacted with BMP alone; and
(h) detecting the presence of neutralizing antibodies in said sample if the amount determined in step (f) is less than or greater than the amount determined in step (g).

2. The method of claim 1, further comprising the steps of:
(i) amplifying a housekeeping gene from said cDNA of step (d) by quantitative real-time polymerase chain reaction (QPCR);
(j) determining the amount of said housekeeping gene amplified in step (i) in said cells; and
(k) normalizing the amount of said gene determined in step (f) with the amount of said housekeeping gene determined in step (j).

3. The method of claim 1 or 2, wherein said BMP is selected from the group consisting of OP-1 (BMP-7), OP-2, OP-3, BMP-2, BMP-3, BMP-4, BMP-5, BMP-6, BMP-8, BMP-9, BMP-10, BMP-11, BMP-12, BMP-13, BMP-15, BMP-16, BMP-17, BMP-18, DPP, Vg1, Vgr, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, NODAL, UNIVIN, SCREW, ADMP, NEURAL, and fragments thereof.

4. The method of claim 3, wherein said BMP is OP-1 (BMP-7).

5. The method of claim 1 or 2, wherein said sample is serum.

6. The method of claim 5, wherein said serum is human serum.

7. The method of claim 1 or 2, wherein said BMP-responsive cell is selected from the group consisting of A549, SAOS-2, ROS and CSC12.

8. The method of claim 7, wherein said BMP-responsive cell is a A549 cell.

9. The method of claim 1 or 2, wherein said gene is an early gene.

10. The method of claim 9, wherein said early gene is selected from the group consisting of Id-1, Id-2, Id-3, Id-4, Msx2, Dlx2, Dlx3, Dlx5, Noggin, Smad6, Smad7 and Runx2.

11. The method of claim 10, wherein said early gene is Id-1.

12. The method of claim 1 or 2, wherein said gene is a late gene.

13. The method of claim 1 or 2, wherein said late gene is selected from the group consisting of HEY1, DIO2, ADAMTS9, HAS3, FGFR3, MFI2, CHI3L1, NOG, BAMBI, GREM1, GREM2 and SOST.

14. The method of claim 1 or 2, wherein said sample is contacted with a BMP in step (a) for at least 30 minutes.

15. The method of claim 1 or 2, wherein said sample from step (b) is incubated with a BMP-responsive cell for about 3 hours.

16. The method of claim 1 or 2, wherein the antibody is specific for OP1 (BMP-7).

17. The method of claim 1 or 2, wherein said sample is subjected to at least one prescreening assay prior to performing steps (a)-(h).

18. The method of claim 17, wherein said prescreening assay is any assay that is capable of detecting neutralizing antibodies to a bone morphogenetic protein (BMP).

19. The method of claim 18, wherein said prescreening assay is selected from the group consisting of immunoassay and cell-based assay.

20. The method of claim 19, wherein said immunoassay is an enzyme-linked-immunosorbent assay (ELISA).

21. The method of claim 19, wherein said cell-based assay involves activation of a reporter gene.

22. The method of claim 21, wherein said reporter gene is a luciferase gene.

23. The method of claim 22, wherein said luciferase gene is linked to a promoter of a gene that is capable of being modulated by exposure to a BMP.

24. The method of claim 23, wherein said gene is an early or late gene.

25. The method of claim 23, wherein said gene is selected from the group consisting of Id-1, Id-2, Id-3, Id-4, Msx2, Dlx2, Dlx3, Dlx5, Noggin, Smad6, Smad7, Runx2, fibromodulin, Hey 1 and SFRP-2.

26. The method of claim 19, wherein said cell-based assay involves monitoring the activity of alkaline phosphatase.

27. The method of claim 26, wherein said alkaline phosphatase activity is monitored in rat osteosarcoma (ROS) cells.
